SEQUENCE LISTING (12 sequences)

SEQ ID NO: 1 - DNA, 609 bp, Homo sapiens
  CDS (1)...(609)
cgggctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctcagggtgctccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcgcaggaccgggc

SEQ ID NO: 2 - protein, 203 aa, Homo sapiens
RAAGGILHLELLVAVGPDVFQAHQEDTERYVLTNLNIGAELLRDPSLGAQFRVHLVKMVILTEPQGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSAGPG

SEQ ID NO: 3 - DNA, 609 bp, Artificial Sequence
  Degenerate sequence
  misc_feature (1)...(609): n = A,T,C or G
mgngcngcnggnggnathytncayytngarytnytngtngcngtnggnccngaygtnttycargcncaycargargayacngarmgntaygtnytnacnaayytnaayathggngcngarytnytnmgngayccnwsnytnggngcncarttymgngtncayytngtnaaratggtnathytnacngarccncarggngcnccnaayathacngcnaayytnacnwsnwsnytnytnwsngtntgyggntggwsncaracnathaayccngargaygayacngayccnggncaygcngayytngtnytntayathacnmgnttygayytngarytnccngayggnaaymgncargtnmgnggngtnacncarytnggnggngcntgywsnccnacntggwsntgyytnathacngargayacnggnttygayytnggngtnacnathgcncaygarathggncaywsnttyggnytngarcaygayggngcnccnggnwsnggntgyggnccnwsnggncaygtnatggcnwsngayggngcngcnccnmgngcnggnytngcntggwsnccntgywsnmgnmgncarytnytnwsngcnggnccnggn

SEQ ID NO: 4 - DNA, 144 bp, Homo sapiens
  CDS (1)...(144)
tggtctagctggggtccccgaagtccttgctcccgctcctgcggaggatgtggtcaccaggaggcggcagtgcaacaaccccaggtaccgcagggagggcttttctgccaaggaatgaagctgggtgggggctgggggacttgc

SEQ ID NO: 5 - protein, 48 aa, Homo sapiens
WSSWGPRSPCSRSCGGCGHQEAAVQQPQVPQGGLFCQGMKLGGGWGTC

SEQ ID NO: 6 - DNA, 144 bp, Artificial Sequence
  Degenerate sequence
  misc_feature (1)...(144): n = A,T,C or G
tggwsnwsntggggnccnmgnwsnccntgywsnmgnwsntgyggnggntgyggncaycargargcngcngtncarcarccncargtnccncarggnggnytnttytgycarggnatgaarytnggnggnggntggggnacntgy

SEQ ID NO: 7 - DNA, 177 bp, Homo sapiens
  CDS (1)...(177)
tggcagtacaagctggcggcctgcagcgtgagctgtgggagaggggtcgtgcggaggatcctgtattgtgcccgggcccatggggaggacgatggtgaggagatcctgttggacacccagtgccaggggctgcctcgcccggaaccccaggaggcctgcagcctggagccctgccca

SEQ ID NO: 8 - protein, 59 aa, Homo sapiens
WQYKLAACSVSCGRGVVRRILYCARAHGEDDGEEILLDTQCQGLPRPEPQEACSLEPCP

SEQ ID NO: 9 - DNA, 177 bp, Artificial Sequence
  Degenerate sequence
  misc_feature (1)...(177): n = A,T,C or G
tggcartayaarytngcngcntgywsngtnwsntgyggnmgnggngtngtnmgnmgnathytntaytgygcnmgngcncayggngargaygayggngargarathytnytngayacncartgycarggnytnccnmgnccngarccncargargcntgywsnytngarccntgyccn

SEQ ID NO: 10 - DNA, 3363 bp, Homo sapiens
  CDS (1)...(3363)
  misc_feature (1)...(3363): n = A,or G
gcaggcctgtcccattccatactgaccagattcccagtcaccaaggccccctctcactccgctccactcctcgggctggctctcctgaggatgcaccagcgtcacccccgggcaagatgccctcccctctgtgtggccggaatccttgcctgtggctttctcctgggctgctggggaccctcccatttccagcagagttgtcttcaggctttggagccacaggccgtgtcttcttacttgagccctggtgctcccttaaaaggccgccctccttcccctggcttccagaggcagaggcagaggcagaggcgggctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctcagggtgctccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcgcaggaccgggcgcgctgcgtgtggggacccgccgcggcctcaaacccgggttccgcggggcacccgccggatggcgcagccttggcctctactacagcgccaacgagcagtgccacgtcgcgttcggccccccagggtgtcgcctgcaccttcgccagggagcaccttgccaggccctctcctgccacacagacccgctggaccaaagcagctgcagccgcctcctcgttcctctcctggatgggacagaatgtggcgtggagaaggtgcatgggcgctggtctagctggggtccccgaagtccttgctcccgctcctgcggaggatgtggtcaccaggaggcggcagtgcaacaaccccaggtaccgcagggagggcttttctgccaaggaatgaagctgggtgggggctgggggacttgcccctcctgctcggttcaggacaccctttttcactctgccctcccaggggatgctctgtgcagacacatgtgccgggccattggcgagagcttncatcatgaagcgtgggagacagcttcctcgaatgggacccggtgtatgccaagtggcccccgggaggacgggaccctgagcctgtgtgtgtcgggcagctgcagggttaggggatgtgacggaaggatggactcccagcaggtatgggacaggtgccaggtgtgtggtggggacaacagcacgtgcagcccacggaagggctctttcacagctggcagagcgagagaatatgtcacgtttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtaccagtgtgtaaaaaagcagattcccgggtcctctgcatattccctgaatcaggacttccctgtgttgggcctgagaaaccgcaccgtaaccaacacaggcttgcggcactggccagatgtgggcatcgagggggcaggtctgatggagctgcgtttcctgtgcatggactctgccctcagggtgcctgtccaggaagagctgtgtggcctggcaagcaagcctgggagccggcgggaggtctgccaggctgtcccgtgccctgctcggtggcagtacaagctggcggcctgcagcgtgagctgtgggagaggggtcgtgcggaggatcctgtattgtgcccgggcccatggggaggacgatggtgaggagatcctgttggacacccagtgccaggggctgcctcgcccggaaccccaggaggcctgcagcctggagccctgcccacctaggtggaaagtcatgtcccttggcccatgttcggccagctgtggccttggcactgctagacgctcggtggcctgtgtgcagctcgaccaaggccaggacgtggaggtggacgaggcggcctgtgcggcgctggtcgcggcccgaggccagttgtcccctgtctcattgccgactgcacctaccgctggcatgttggcacctggatggaggcgtgggagtgctggaccctcactgccctgccgcttcctaggggacatgttgctgctttggggccggctcacctggaggaagatgtgcaggaagctgttggacatgactttcagctccaagaccaacacgctggtgatccgggacacccacagcttgaggaccacagcgttccatcgggcagcaggtgctctaactgggagtcagagagcagccaggctgaggatggagttcagcgagggcttcctgaaggctcaggccagcctgcggggccagtactggaccctccaatcatggcttgcgcgagtctctggcctcttcaactgcatcaccatccaccctctgaacattgcggccggcgtgtggatgatcatgaatgccttcatcttgttgctgtgtgaggcgcccttctgctgccagttcatcgagtttgcaaacacagtggcggagaaggtggacccgctgcgctcctggcagaaggctgtcttctactgcggctgccaaaacgtgccccagtggttctgcgcccaggaacttcagctgtcgctgtgccgtagtcactggaaggttcagaggtatatgagcatgtgtggcagcatccgtgcagtggagagaaagtgggaagcgtctggaattttggtccgtccactgggagttgttaaccagatgatagatggcggtcgttcccatcgtcatcagcctgaccctgaccaacgctgcctgggcaacgccattcgccttttgctacgggggctgctgtacggactctctgctctgggcaaaaagggcgatgcgatctcctatgccaggatccagcagcagaggcagcaggcggatgaggagaagctcgcggagaccctggagggggagctgtgaaataaa

SEQ ID NO: 11 - protein, 1120 aa, Homo sapiens
  VARIANT (1)...(1120): Xaa = Any Amino Acid
AGLSHSILTRFPVTKAPSHSAPLLGLALLRMHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPQGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSAGPGALRVGTRRGLKPGFRGAPAGWRSLGLYYSANEQCHVAFGPPGCRLHLRQGAPCQALSCHTDPLDQSSCSRLLVPLLDGTECGVEKVHGRWSSWGPRSPCSRSCGGCGHQEAAVQQPQVPQGGLFCQGMKLGGGWGTCPSCSVQDTLFHSALPGDALCRHMCRAIGESXHHEAWETASSNGTRCMPSGPREDGTLSLCVSGSCRVRGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYQCVKKQIPGSSAYSLNQDFPVLGLRNRTVTNTGLRHWPDVGIEGAGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVAARGQLSPVSLPTAPTAGMLAPGWRRGSAGPSLPCRFLGDMLLLWGRLTWRKMCRKLLDMTFSSKTNTLVIRDTHSLRTTAFHRAAGALTGSQRAARLRMEFSEGFLKAQASLRGQYWTLQSWLARVSGLFNCITIHPLNIAAGVWMIMNAFILLLCEAPFCCQFIEFANTVAEKVDPLRSWQKAVFYCGCQNVPQWFCAQELQLSLCRSHWKVQRYMSMCGSIRAVERKWEASGILVRPLGVVNQMIDGGRSHRHQPDPDQRCLGNAIRLLLRGLLYGLSALGKKGDAISYARIQQQRQQADEEKLAETLEGELNK

SEQ ID NO: 12 - DNA, 2379 bp, Artificial Sequence
  Degenerate sequence
  misc_feature (1)...(2379): n = A,T,C or G
gcnggnytnwsncaywsnathytnacnmgnttyccngtnacnaargcnccnwsncaywsngcnccnytnytnggnytngcnytnytnmgnatgcaycarmgncayccnmgngcnmgntgyccnccnytntgygtngcnggnathytngcntgyggnttyytnytnggntgytggggnccnwsncayttycarcarwsntgyytncargcnytngarccncargcngtnwsnwsntayytnwsnccnggngcnccnytnaarggnmgnccnccnwsnccnggnttycarmgncarmgncarmgncarmgnmgngcngcnggnggnathytncayytngarytnytngtngcngtnggnccngaygtnttycargcncaycargargayacngarmgntaygtnytnacnaayytnaayathggngcngarytnytnmgngayccnwsnytnggngcncarttymgngtncayytngtnaaratggtnathytnacngarccncarggngcnccnaayathacngcnaayytnacnwsnwsnytnytnwsngtntgyggntggwsncaracnathaayccngargaygayacngayccnggncaygcngayytngtnytntayathacnmgnttygayytngarytnccngayggnaaymgncargtnmgnggngtnacncarytnggnggngcntgywsnccnacntggwsntgyytnathacngargayacnggnttygayytnggngtnacnathgcncaygarathggncaywsnttyggnytngarcaygayggngcnccnggnwsnggntgyggnccnwsnggncaygtnatggcnwsngayggngcngcnccnmgngcnggnytngcntggwsnccntgywsnmgnmgncarytnytnwsngcnggnccnggngcnytnmgngtnggnacnmgnmgnggnytnaarccnggnttymgnggngcnccngcnggntggmgnwsnytnggnytntaytaywsngcnaaygarcartgycaygtngcnttyggnccnccnggntgymgnytncayytnmgncarggngcnccntgycargcnytnwsntgycayacngayccnytngaycarwsnwsntgywsnmgnytnytngtnccnytnytngayggnacngartgyggngtngaraargtncayggnmgntggwsnwsntggggnccnmgnwsnccntgywsnmgnwsntgyggnggntgyggncaycargargcngcngtncarcarccncargtnccncarggnggnytnttytgycarggnatgaarytnggnggnggntggggnacntgyccnwsntgywsngtncargayacnytnttycaywsngcnytnccnggngaygcnytntgymgncayatgtgymgngcnathggngarwsnytncaycaygargcntgggaracngcnwsnwsnaayggnacnmgntgyatgccnwsnggnccnmgngargayggnacnytnwsnytntgygtnwsnggnwsntgymgngtnmgnggntgygayggnmgnatggaywsncarcargtntgggaymgntgycargtntgyggnggngayaaywsnacntgywsnccnmgnaarggnwsnttyacngcnggnmgngcnmgngartaygtnacnttyytnacngtnacnccnaayytnacnwsngtntayathgcnaaycaymgnccnytnttyacncayytngcngtnmgnathggnggnmgntaygtngtngcnggnaaratgwsnathwsnccnaayacnacntayccnwsnytnytngargayggnmgngtngartaymgntgygtnaaraarcarathccnggnwsnwsngcntaywsnytnaaycargayttyccngtnytnggnytnmgnaaymgnacngtnacnaayacnggnytnmgncaytggccngaygtnggnathgarggngcnggnytnatggarytnmgnttyytntgyatggaywsngcnytnmgngtnccngtncargargarytntgyggnytngcnwsnaarccnggnwsnmgnmgngargtntgycargcngtnccntgyccngcnmgntggcartayaarytngcngcntgywsngtnwsntgyggnmgnggngtngtnmgnmgnathytntaytgygcnmgngcncayggngargaygayggngargarathytnytngayacncartgycarggnytnccnmgnccngarccncargargcntgywsnytngarccntgyccnccnmgntggaargtnatgwsnytnggnccntgywsngcnwsntgyggnytnggnacngcnmgnmgnwsngtngcntgygtncarytngaycarggncargaygtngar

(19) United States
(12) Patent Application Publication
(10) Pub. No.: US 2002/0142439 A1
(43) Pub. Date: Oct. 3, 2002

(54) Anti-angiogenic intestinal peptides, zdint5

(76) Inventors: James L. Holloway, Seattle, WA (US); Paul O. Sheppard, Granite Falls, WA (US); Gayle Yamamoto, Seattle, WA (US)

Correspondence Address: Robyn Adams, ZymoGenetics, Inc., 1201 Eastlake Avenue East, Seattle, WA, 98102, US

(21) Appl. No.: 09/781,080

(22) Filed: Feb. 9, 2001

Related U.S. Application Data
(60) Provisional application No. 60/181,511, filed on Feb. 10, 2000.

(51) Int. Cl. C12N 9/64; C07H 21/04; C12P 21/02; C12N 5/06

(52) U.S. Cl. 435/226; 435/69.1; 435/325; 435/320.1; 536/23.2

(57) ABSTRACT

The present invention relates to polynucleotide and polypeptide molecules, and variants thereof, for zdint5, a novel member of the Disintegrin Proteases. The polypeptides, and polynucleotides encoding them, are cell-cell interaction modulating and may be used for delivery and therapeutics. The present invention also includes antibodies to the zdint5 polypeptides.

This application is related to Provisional Application No. 60/181,51 filed on Feb. 10, 2000. Under 35 U.S.C. §119(e)(1), this application claims benefit of said Provisional Application.

### BACKGROUND OF THE INVENTION

Proteins involved in extracellular matrix formation and degradation, particularly proteolytic proteins, are critical in establishing tissue architecture during development and in tissue degradation in a variety of diseases including cancer, arthritis, Alzheimer's disease and a variety of inflammatory conditions. Specifically relevant to extracelluar proteolysis are the zinc metalloproteases which have been identified in ADAMs (A Disintegrin and Metalloprotease), MMPs (Matrix Metalloproteases), MDCs (Metalloprotease-Disintegrin-Cysteine-rich proteins), and SVMPs (Snake Venom Metalloprotease Proteins). The cleavage activities of these proteins include proteolysis for matrix molecules as well as non-matrix molecules including tumor necrosis factor α. See Hurskainen, T. et al., J. Biol. Chem. 274: 25555-25563, 1999.

Thrombospondin-1 (TSP1) is an extracellular matrix associated protein that has the ability to inhibit angiogenesis in vivo. TSP1 blocks capillary-like tube formation and endothelial cell proliferation in vitro. The anti-angiogenic activity of TSP1 has been mapped to a region which contains three type 1 repeats. Recombinant and proteolytic fragments of these repeats exhibited angio-inhibitory activity in the rabbit corneal pocket and chorioallantoic membrance assays. Peptides derived from the second and third type 1 repeats of TSP1 inhibit endothelial cells and suppress tumor growth when injected systemically. See Vazquez, F. et al., J. Biol. Chem. 274: 23349-23357, 1999.

Related to the ADAMs are the ADAM-TS (A Metalloprotease and Disintegrin with Thrombospondin-1 repeats), and METHs (Metalloprotease and Thrombospondin-1 repeat proteins). ADAMTS-1 is characterized as a disintegrin and metalloprotease with thrombospondin motifs and is an inflammation-associated gene that has also identified as a cachexia tumor slelective gene (Kuno, K. et al., J. Biol. Chem. 272: 556-562, 1997). METH-1 is a combination of metalloprotease and thrombospondin domains and inhibits angiogenesis (Vasquez, ibid.) For an additional review of the ADAMTS protein family, see Tang, B. L., et al., FEBS Letters 445: 223-225, 1999. An additional ADAMTS family member, ADAMTS, ADAMTS-2 has been implicated as a cartilage “aggrecanase”; see Flannery, C. R., et al., Bioc. and Bioph. Res. Comm. 260:318-322, 1999.

Members of the ADAMs/ADAMS-TS/MDCs/SVMPs/METH family of proteins which have been shown to be therapeutically useful include eptifibatide (Integrilin®, made by COR Therapeutics, Inc. and Key Pharmaceuticals, Inc.) which is useful as an anti-clotting agent for acute coronary syndrome, and contortrostatin, which inhibits β₁Integrin-mediated human metastatic melanoma cell adhesion and blocks experimental metastasis (Trikha, M. et at., Cancer Research 54: 4993-4998, 1994) and inhibits platelet aggregation (Clark, E. A. et al., J. Biol. Chem. 269 (35):21940-21943, 1994).

The present invention provides a novel member of the ADAMs/ADAMS-TS/MDCs/SVMPs/METH family and related compositions whose uses will be apparent to those skilled in the art from the teachings herein.

### SUMMARY OF THE INVENTION

Within one aspect the invention provides an isolated polypeptide comprising the amino acid sequence as shown in SEQ ID NO:2. Within an embodiment, the polypeptide comprises an amino acid sequence that is at least 80% identical to the amino acid sequence as shown in SEQ ID NO:2. Within additional embodiments, the amino acid sequence is at least 90%, 95%, 98%, or 99% identical to the amino acid sequence as shown in SEQ ID NO:2.

Within another aspect the invention provides an isolated polypeptide comprising the amino acid sequence as shown in SEQ ID NO:5. Within an embodiment, the polypeptide comprising an amino acid sequence that is at least 80% identical to the amino acid sequence as shown in SEQ ID NO:5. Within additional embodiments, the amino acid sequence is at least 90%, 95%, 98%, or 99% identical to the amino acid sequence as shown in SEQ ID NO:5.

Within another aspect the invention provides an isolated polypeptide comprising the amino acid sequence as shown in SEQ ID NO:8. Within an embodiment, the polypeptide comprising an amino acid sequence that is at least 80% identical to the amino acid sequence as shown in SEQ ID NO:8. Within additional embodiments, the amino acid sequence is at least 90%, 95%, 98%, or 99% identical to the amino acid sequence as shown in SEQ ID NO:8.

Within another aspect the invention provides an isolated polypeptide comprising the amino acid sequence as shown in SEQ ID NO:11. Within an embodiment, the polypeptide comprising an amino acid sequence that is at least 80% identical to the amino acid sequence as shown in SEQ ID NO:11. Within additional embodiments, the amino acid sequence is at least 90%, 95%, 98%, or 99% identical to the amino acid sequence as shown in SEQ ID NO:11.

Within another aspect the invention provides an isolated polypeptide selected from the group consisting of: a) a polypeptide comprising the amino acid sequence as shown in SEQ ID NO:2; b) a polypeptide comprising the amino acid sequence as shown in SEQ ID NO:5; c) a polypeptide comprising the amino acid sequence as shown in SEQ ID NO8; and d) a polypeptide comprising the amino acid sequence as shown in SEQ ID NO11; wherein the polypeptide is operably linked via a peptide bond or polypeptide linker to a second polypeptide selected from the group consisting of maltose binding protein, an immunoglobulin constant region, and a polyhistidine tag.

Within another aspect is provided an isolated polynucleotide encoding a fusion protein comprising a first polypeptide segment and a second polypeptide segment, wherein the first polypeptide segment comprises a protease domain and the second polypeptide segment comprises a polypeptide selected from the group consisting of: a polypeptide comprising residues 1 to 48 of SEQ ID NO:5; and a polypeptide comprising residues 1 to 59 of SEQ ID NO:8; wherein the first polypeptide segment is positioned amino-terminally to the second polypeptide segment.

Within another aspect the invention provides an isolated polynucleotide encoding a fusion protein comprising a first polypeptide segment and a second polypeptide segment, wherein the first polypeptide segment comprises residues 1 to 203 of SEQ ID NO:2, and the second polynucleotide segment encodes a second polypeptide that is a TSP1-like domain, and wherein the first polynucleotide segment is positioned amino-terminally to the second polynucleotide segment.

Within another aspect the invention provides an expression vector comprising the following operably linked elements: a) a transcription promoter; b) a DNA segment encoding a polypeptide, wherein the amino acid sequnce of the polypeptide comprises the amino acid sequence selected from the group consisting of: the amino acid sequence as shown in SEQ ID NO:2; the amino acid sequence as shown in SEQ ID NO:5; the amino acid sequence as shown in SEQ ID NO:8; and the amino acid sequence as shown in SEQ ID NO:2;and c) a transcription terminator. Within an embodiment is provided a cultured cell into which has been introduced an expression vector, wherein said cell expresses the polypeptide encoded by the DNA segment. Within another embodiment the invention provides a method of producing a polypeptide comprising culturing the cell, whereby said cell expresses the polypeptide encoded by the DNA segment, and recovering the polypeptide. Within a further embodiment is provided the polypeptide made by the method.

Within another embodiment is provided a method for modulating extracellular matrix interactions by combining a polypeptide with cells, wherein the polypeptide is selected from the group consisting of: a) a polypeptide comprising the amino acid sequence as shown in SEQ ID NO:2; b) a polypeptide comprising the amino acid sequence as shown in SEQ ID NO:5; c) a polypeptide comprising the amino acid sequence as shown in SEQ ID NO8; and d) a polypeptide comprising the amino acid sequence as shown in SEQ ID NO11. Within an embodiment the method for modulating extracellular matrix interactions, whereby the cells are derived from tissues selected from the group consisting of: a) tissues from colon; b) tissues from small intestine; c) tissues from testes; and d) tissues from lung.

Within another aspect the invention provides a method of producing an antibody to the polypeptide made by the method of claim 31 comprising the following steps: inoculating an animal with the polypeptide such that the polypeptide elicits an immune response in the animal to produce the antibody; and isolating the antibody from the animal. Within an embodiment the antibody specifically binds to a polypeptide selected from the group consisting of: a) a polypeptide comprising the amino acid sequence as shown in SEQ ID NO:2; b) a polypeptide comprising the amino acid sequence as shown in SEQ ID NO:5; c) a polypeptide comprising the amino acid sequence as shown in SEQ ID NO8; and d) a polypeptide comprising the amino acid sequence as shown in SEQ ID NO11.

Within another aspect, the invention provides an isolated polypeptide comprising at least 11 contiguous amino acid residues of SEQ ID NO:11. Within an embodiment, the isolated polypeptide comprises at least 15 contiguous amino aicds of SEQ ID NO:11. Within another embodiment, the isolated polypeptide comprises at least 30 contiguous amino aicds of SEQ ID NO:11. Within another embodiment, the 11 contiguos amino acid residues are from the group consisting of: (a) SEQ ID NO:2; (b) SEQ ID NO:5; and (c)SEQ ID NO:8. Within another embodiment, the isolated polypeptide is between 48 and 1120 amino acids in length. Within another embodiment, at least nine of the contiguous amino acid residues are operably linked via a peptide bond or polypeptide linker to a second polypeptide selected from the group consisting of maltose binding protein, an immunoglobulin constant region, and a polyhistidine tag.

Within another aspect, the invention provides an isolated polynucleotide encoding a polypeptide comprising at least 11 contiguous amino acid residues of SEQ ID NO:11, wherein the contiguous sequence of 11 amino acids is selected from the group consisting of: (a) a polypeptide comprising the amino acids of SEQ ID NO:5; (b) a polypeptide comprising the amino acids of SEQ ID NO:8; and (c) a polypeptide comprising the amino acids of SEQ ID NO:11. Within another embodiment, the polypeptide molecule is between 48 and 1120 amino acids in length.

Within another aspect, the invention provides an isolated polynucleotide encoding a fusion protein comprising a first polypeptide segment and a second polypeptide segment, wherein the first polypeptide segment comprises a protease domain and the second polypeptide segment comprises a polypeptide molecule selected from the group consisting of: (a) a polypeptide comprising residues 1 to 48 of SEQ ID NO:5; and (b) a polypeptide comprising residues 1 to 59 of SEQ ID NO:8; wherein the first polypeptide segment is positioned amino-terminally to the second polypeptide segment.

Within another aspect, the invention provides an isolated polynucleotide encoding a fusion protein comprising a first polypeptide segment and a second polypeptide segment, wherein the first polypeptide segment comprises residues 1 to 203 of SEQ ID NO:2, and the second polynucleotide segment encodes a second polypeptide that is a TSP1-like domain, and wherein the first polynucleotide segment is positioned amino-terminally to the second polynucleotide segment.

Within another aspect, the invention provides an expression vector comprising the following operably linked elements: a) a transcription promoter; b) a DNA segment encoding the polypeptide of comprising at least 11 contiguous amino acid residues of SEQ ID NO:11; and c) a transcription terminator. Within an embodiment, the DNA segment further encodes an affinity tag. Within another embodiment, the invention provides a cultured cell into which has been introduced the expression vector, wherein said cell expresses the polypeptide encoded by the DNA segment. Within another embodiment, is provided a method of producing a polypeptide comprising culturing the cell, whereby said cell expresses the polypeptide encoded by the DNA segment, and recovering the polypeptide. Within another embodiment,the invention provides the polypeptide made by the method.

Within another aspect, the invention provides a method for modulating extracellular matrix interactions by combining the polypeptide comprising at least 11 contiguous amino acid residues of SEQ ID NO:11 with cells in vivo or in vitro. Within another embodiment, the cells are derived from tissues selected from the group consisting of: a) tissues from colon; b) tissues from small intestine; c) tissues from testes; and d) tissues from lung. Within another aspect, the invention provides a method of producing an antibody to the polypeptide comprising the following steps: inoculating an animal with the polypeptide of claim 15 such that the polypeptide elicits an immune response in the animal to produce the antibody; and isolating the antibody from the animal. Within another embodiment, is provided an antibody produced by the methodwhich binds to a protein comprising the a polypeptide wherein the polypeptide is selected from the group consisting of: (a) SEQ ID NO:2; (b) SEQ ID NO:5; (c) SEQ ID NO:8; and (d) SEQ ID NO:11.

These and other aspects of the invention will become evident upon reference to the following detailed description of the invention and attached drawings.

### DETAILED DESCRIPTION OF THE INVENTION

Prior to setting forth the invention in detail, it may be helpful to the understanding thereof to define the following terms:

The term “affinity tag” is used herein to denote a polypeptide segment that can be attached to a second polypeptide to provide for purification of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu-Glu affinity tag (Grussenmeyer et al., Proc. Natl. Acad. Sci. USA 82:7952-4, 1985) (SEQ ID NO:7), substance P, Flag™ peptide (Hopp et al., Biotechnology 6:1204-1210, 1988), streptavidin binding peptide, maltose binding protein (Guan et al., Gene 67:21-30, 1987), cellulose binding protein, thioredoxin, ubiquitin, T7 polymerase, or other antigenic epitope or binding domain. See, in general, Ford et al., Protein Expression and Purification 2: 95-107, 1991. DNAs encoding affinity tags and other reagents are available from commercial suppliers (e.g., Pharmacia Biotech, Piscataway, N.J.; New England Biolabs, Beverly, Mass.; Eastman Kodak, New Haven, Conn.).

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “complements of a polynucleotide molecule” is a polynucleotide molecule having a complementary base sequence and reverse orientation as compared to a reference sequence. For example, the sequence 5′ ATGCACGGG 3′ as complementary to 5′ CCCGTGCAT 3′.

The term “corresponding to”, when applied to positions of amino acid residues in sequences, means corresponding positions in a plurality of sequences when the sequences are optimally aligned.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

“Operably linked” means that two or more entities are joined together such that they function in concert for their intended purposes. When referring to DNA segments, the phrase indicates, for example, that coding sequences are joined in the correct reading frame, and transcription initiates in the promoter and proceeds through the coding segment(s) to the terminator. When referring to polypeptides, “operably linked” includes both covalently (e.g., by disulfide bonding) and non-covalently (e.g., by hydrogen bonding, hydrophobic interactions, or salt-bridge interactions) linked sequences, wherein the desired function(s) of the sequences are retained.

The term “ortholog” or “species homolog”, denotes a polypeptide or protein obtained from one species that is the functional counterpart of a polypeptide or protein from a different species. Sequence differences among orthologs are the result of speciation.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nt in length.

A “polypeptide” is a polymer of amino acid residues joined by peptide bonds, whether produced naturally or synthetically. Polypeptides of less than about 10 amino acid residues are commonly referred to as “peptides”.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” denotes a cell-associated protein that binds to a bioactive molecule (i.e., a ligand) and mediates the effect of the ligand on the cell. Membrane-bound receptors are characterized by a multi-domain or multi-peptide structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

A “segment” is a portion of a larger molecule (e.g., polynucleotide or polypeptide) having specified attributes. For example, a DNA segment encoding a specified polypeptide is a portion of a longer DNA molecule, such as a plasmid or plasmid fragment, that, when read from the 5′ to the 3′ direction, encodes the sequence of amino acids of the specified polypeptide.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

A discussion of the domain structure of some members of the ADAM-TS and METH family members will aid to illustrate the present invention in better detail. The secretory peptide has been described above.

The propeptide domain is usually amino-terminal to the metalloprotease domain and is can act as an inhibitor for the metalloprotease domain (presumably via a cysteine-switch mechanism), such that the metalloprotease domain is activated in certain circumstances. This inhibition can be by blocking the active site of the metalloprotease domain.

The protease domain may be active or inactive. Some members of the disintegrin family have “active” zinc catalytic sites, which may be regulated by a “cysteine-switch” in the cysteine-rich domain. An example of a family member which has an “active” protease domain is ADAM-TS 1, which is thought to be involved in the inflammatory process through a processing of proteins in the extracellular matrix.

Members of this family, which do not have such a catalytic site, include, for example, ADAM 11, which may be involved in tumor suppression. Other protein families, which are known to have inactive protease domains, are the serine proteases.

The adhesion (disintegrin) domain binds integrins or cell surface receptors which can be located on the surface of a multitude of cells, depending on the specificity of the disintegrin. The predicted binding site within this disintegrin domain is often an amino acid loop comprising about 13 to 14 amino acids. See Wolfsberg and White, supra) The conformation of this sequence upon folding results in a hairpin loop presenting an amino acid sequence at its tip. This sequence is often “RGD”, but may be substituted by a variety of other amino acid residues (Wolfsberg and White, supra; and Jia, J. Biol. Chem. 272:13094-13102, 1997). Receptors for the specific classes of disintegrin domains can recognize a multitude of disintegrin binding loop sequences. Disintegrin domains have been shown to be responsible for cell-cell interactions, including inhibition of platelet aggregation by binding GPIIb/IIIa (fibronectin receptor) and/or GPIa/IIa (collagen receptor). The METH proteins have two disintegrin domains.

Many disintegrin family members have a fusion domain, a relatively hydrophobic domain of about 23 amino acids. This domain is present within some of the ADAM family members, and has been shown to be involved in cell-cell fusion, and particularly in sperm/egg fusion, and muscle fusion.

The cysteine-rich domain varies in the ADAMs/ADAMS-TS/MDCs/SVMPs/METH-like family members and is believed to be involved in structurally presenting the integrin-binding region to integrins. For the disintegrin-like members of this family, the cysteine-rich domain may also be necessary for secondary structure conformation of the polypeptide, specifically, disulfide bonding between the disintegrin domain and the cysteine domain.

Many ADAMs/MDCs/SVMPs family members have a transmembrane domain, which acts to anchor the polypeptide to the cell membrane. In the case of the METH proteins, the polypeptide is thought to be anchored via the binding of the TSP1-like domains to the extracellular matrix. Thus METH-1 and METH-2 proteins have been shown to be effective inhibitors of angiogenesis. Membrane-anchored ADAMs/MDCs/SVMPs family members can be involved in a process called “protein ectodomain shedding” wherein the metalloprotease domain cleaves extracellular domain(s) of another protein. In these cases, the metalloprotease can be active on the cell surface itself, as in the case of fertilin (ADAMs 1 and 2), or TACE (ADAM 17), or the metalloprotease can act intracellularly in the secretory pathway as has been described for KUZ and ADAM 10 (Blobel, C. P., supra; and Lammich, S. et al., Proc. Natl. Acad. Sci. USA 96:3922-3927, 1999, respectively). These membrane-anchored metalloproteases are likely to be active in the tissues where their genes are transcribed, in which cases they can be acting in cis, on other proteins bound to the same cell surface, in trans, on proteins bound to other cell surfaces, or on other proteins which are not membrane bound. Additionally the membrane anchor itself can be cleaved resulting in a soluble form of the metalloprotease/disintegrin which can be active at other sites in the body.

The cytoplasmic, or signaling, domain of the ADAMs/MDCs/SVMPs family members tends to be conserved in length and sites for phosphorylation. However, beyond that they tend to be unique in amino acid composition. Some disintegrin family members may signal by binding to the SH3 domain of Abl, Src, and/or Src-related SH3 domains.

The thrombospondin-like (TSP-like ) domain is located at the carboxyl terminal of the protein. Multiple TSP-like domains can be present. For example, METH-1 has three TSP-like domains, and another METH homolog METH-2 (Vasquez, ibid) has two TSP-like domains. Thrombospondin-1 is a modular protein that associates with the extracellular matrix and has the ability to inhibit angiogenesis in vivo. Under culture conditions, thrombospondin-1 blocks capillary-like formation and endothelial cell proliferation. Both METH-1 and METH-2 have also been shown to inhibit angiogenesis in the cornea pocket and CAM assays (Vasquez, ibid).

The present invention is based upon the discovery of novel domains of a member of the METH subfamily of proteins designated zdint5. Domains of zdint5 include: a metalloprotease domain, and two TSP1-like domains. The polynucleotide and polypeptide sequences for the metalloprotease domain are shown in SEQ ID NOs: 1 and 2, respectively. Within the metalloprotease domain is a zinc-binding motif from residue 151 to residue 161 of SEQ ID NO:2 The polynucleotide and polypeptide sequences for the first TSP1-like domain are shown in SEQ ID NOs: 4 and 5, respectively. The polynucleotide and polypeptide sequences for the second TSP1-like domain are shown in SEQ ID NOs: 7and 8, respectively. The degenerate polynucleotide sequences for the metalloprotease, and the first and second TSP1-like domains are shown in SEQ ID NOs: 3, 6, and 9, respectively. An illustrative example of how these domains can be combined in a protein is shown in SEQ ID NO:10 (polynucleotides), SEQ ID NO:11 (polypeptides ), and SEQ ID NO:12, (degenerate polynucleotides). Amino acid residues 69 as shown in SEQ ID NO:2 and 172 as shown in SEQ ID NO:11; 73 as shown in SEQ ID NO:2 and 176 as shown in SEQ ID NO:11; and 485, 533, 560, 595, and 635 all as shown in SEQ ID NO:11 are potential N-linked glycosylation sites.

Analysis of the tissue distribution of zdint5 can be performed by the Northern blotting technique using Human Multiple Tissue and Master Dot Blots. Such blots are commercially available (Clontech, Palo Alto, Calif.) and can be probed by methods known to one skilled in the art. Also see, for example, Wu W. et al., Methods in Gene Biotechnology, CRC Press LLC, 1997. Additionally, portions of the polynucleotides of the present invention can be identified by querying sequence databases and identifying the tissues form which the sequences are derived. Portions of the polynucleotides of the present invention have been identified in a colon adenocarcinoma cDNA library, a small intestine cDNA library, and a cDNA library made from fetal lung, testis, and B-cells.

Some members of the ADAMs family have alternatively spliced isoforms. An example of alternative splicing is ADAM 12, also known as meltrin α. The truncated form of this molecule, which lacks the propeptide and metalloprotease domains, is associated with ectopic muscle formation in vivo, but not in vitro, indicating that cells expressing this gene produce a growth factor that acts on neighboring progenitor cells.

The present invention provides polynucleotide molecules, including DNA and RNA molecules, that encode the zdint5 polypeptides disclosed herein. Those skilled in the art will readily recognize that, in view of the degeneracy of the genetic code, considerable sequence variation is possible among these polynucleotide molecules. SEQ ID NO:3 is a degenerate DNA sequence that encompasses all DNAs that encode the zdint5 polypeptide of SEQ ID NO:2. SEQ ID NO:6 is a degenerate DNA sequence that encompasses all DNAs that encode the zdint5 polypeptide of SEQ ID NO:5. SEQ ID NO:9 is a degenerate DNA sequence that encompasses all DNAs that encode the zdint5 polypeptide of SEQ ID NO:8. SEQ ID NO:12 is a degenerate DNA sequence that encompasses all DNAs that encode the zdint5 polypeptide of SEQ ID NO:11. Those skilled in the art will recognize that the degenerate sequence of SEQ ID NOs:3, 6, 9 and 12 also provides all RNA sequences encoding SEQ ID NOs:2, 5, 8 and 11, respectively, by substituting U for T. Thus, zdint5 polypeptide-encoding polynucleotides comprising nucleotide 1 to nucleotide 609 of SEQ ID NO:3; comprising nucleotide 1 to nucleotide 144 of SEQ ID NO:6; comprising nucleotide 1 to nucleotide 177 of SEQ ID NO:9; and comprising nucleotide 1 to nucleotide 2379 of SEQ ID NO:12 and their RNA equivalents are contemplated by the present invention. Table 1 sets forth the one-letter codes used within SEQ ID NOs:3, 6, 9 and 12 to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 1

| Nucleotide | Resolution | Nucleotide | Complement |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NOs:3, 6, 9 and 12, encompassing all possible codons for a given amino acid, are set forth in Table 2.

TABLE 2

| | One | | |
|---|---|---|---|
| Amino | Letter | | Degenerate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | • | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequences of SEQ ID NOs:2, 5, 8 and 11. Variant sequences can be readily tested for functionality as described herein.

One of ordinary skill in the art will also appreciate that different species can exhibit “preferential codon usage.” Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequences disclosed in SEQ ID NOs:3, 6, 9 and 12 serve as templates for optimizing expression of polynucleotides in various cell types and species commonly used in the art and disclosed herein. Sequences containing preferential codons can be tested and optimized for expression in various species, and tested for functionality as disclosed herein.

Within preferred embodiments of the invention the isolated polynucleotides will hybridize to similar sized regions of SEQ ID NOs:1, 3, 4, 6, 7, 9 10 and 12 or a sequence complementary thereto under stringent conditions. Polynucleotide hybridization is well known in the art and widely used for many applications, see for example, Sambrook et al., Molecular Cloning: A Laboratory Manual, Second Edition, Cold Spring Harbor, N.Y., 1989; Ausubel et al., eds., Current Protocols in Molecular Biology, John Wiley and Sons, Inc., NY, 1987; Berger and Kimmel, eds., Guide to Molecular Cloning Techniques, Methods in Enzymology, volume 152, 1987 and Wetmur, Crit. Rev. Biochem. Mol. Biol. 26:227-59, 1990. Polynucleotide hybridization exploits the ability of single stranded complementary sequences to form a double helix hybrid. Such hybrids include DNA-DNA, RNA-RNA and DNA-RNA.

As an illustration, a nucleic acid molecule encoding a variant zdint5 polypeptide can be hybridized with a nucleic acid molecule having the nucleotide sequence of SEQ ID NOs: 1, 3, 4, 6, 7, 9, 10 and 12 (or their complements) at 42° C. overnight in a solution comprising 50% formamide, 5× SSC (1× SSC: 0.15 M sodium chloride and 15 mM sodium citrate), 50 mM sodium phosphate (pH 7.6), 5× Denhardt's solution (100× Denhardt's solution: 2% (w/v) Ficoll 400, 2% (w/v) polyvinylpyrrolidone, and 2% (w/v) bovine serum albumin), 10% dextran sulfate, and 20 μg/ml denatured, sheared salmon sperm DNA. One of skill in the art can devise variations of these hybridization conditions. For example, the hybridization mixture can be incubated at a higher temperature, such as about 65° C., in a solution that does not contain formamide. Moreover, premixed hybridization solutions are available (e.g., ExpressHyb™ Hybridization Holution from CLONTECH Laboratories, Inc., Palo Alto, Calif.) according to the manufacturer's instructions.

Following hybridization, the nucleic acid molecules can be washed to remove non-hybridized nucleic acid molecules under stringent conditions, or under highly stringent conditions. Typical stringent washing conditions include washing in a solution of 0.5×-2× SSC with 0.1% sodium dodecyl sulfate (SDS) at 55-65° C. That is, nucleic acid molecules encoding a variant zdint5 polypeptide hybridize with a nucleic acid molecule having the nucleotide sequences of SEQ ID NOs: 1, 3, 4, 6, 7, 9, 10 and 12 (or their complements) under stringent washing conditions, in which the wash stringency is equivalent to 0.5×-2× SSC with 0.1% SDS at 55-65° C., including 0.5× SSC with 0.1% SDS at 55° C., or 2× SSC with 0.1% SDS at 65° C. One of skill in the art can readily devise equivalent conditions, for example, by substituting SSPE for SSC in the wash solution.

The present invention also contemplates zdint5 variant nucleic acid molecules that can be identified using two criteria: a determination of the similarity between the encoded polypeptides with the amino acid sequences of SEQ ID NOs:2, 5, 8 and 11 (as described below), and a hybridization assay, as described above. Such zdint5 variants include nucleic acid molecules (1) that hybridize with a nucleic acid molecule having the nucleotide sequence of SEQ ID NOs: 1, 3, 4, 6, 7, 9, 10 and 12 (or their complements) under stringent washing conditions, in which the wash stringency is equivalent to 0.5×-2× SSC with 0.1% SDS at 55-65° C., and (2) that encode a polypeptide having at least 80%, preferably 90%, more preferably, 95% or greater than 95% sequence identity to the amino acid sequence of SEQ ID NOs:2, 5, 8 or 11. Alternatively, zdint5 variants can be characterized as nucleic acid molecules (1) that hybridize with a nucleic acid molecule having the nucleotide sequence of SEQ ID NOs: 1 or 3 (or their complements) under highly stringent washing conditions, in which the wash stringency is equivalent to 0.1×-0.2× SSC with 0.1% SDS at 50-65° C., and (2) that encode a polypeptide having at least 80%, preferably 90%, more preferably 95% or greater than 95% sequence identity to the amino acid sequence of SEQ ID NOs:2, 5, 8 or 11.

The highly conserved amino acids in the disintegrin domain of zdint5 can be used as a tool to identify new family members. For instance, reverse transcription-polymerase chain reaction (RT-PCR) can be used to amplify sequences encoding the conserved disintegrin domain from RNA obtained from a variety of tissue sources or cell lines. In particular, highly degenerate primers designed from the zdint5 sequences are useful for this purpose.

As previously noted, the isolated polynucleotides of the present invention include DNA and RNA. Methods for preparing DNA and RNA are well known in the art. In general, RNA is isolated from a tissue or cell that produces large amounts of zdint5 RNA. Such tissues and cells are identified by Northern blotting (Thomas, Proc. Natl. Acad. Sci. USA 77:5201, 1980), and include colon, small intestine, fetal lung, testis, and B-cells.

Total RNA can be prepared using guanidine isothiocyante extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-12, 1972). Complementary DNA (cDNA) is prepared from poly(A)⁺ RNA using known methods. In the alternative, genomic DNA can be isolated. Polynucleotides encoding zdint5 polypeptides are then identified and isolated by, for example, hybridization or PCR.

A full-length clone encoding zdint5 can be obtained by conventional cloning procedures. Complementary DNA (cDNA) clones are preferred, although for some applications (e.g., expression in transgenic animals) it may be preferable to use a genomic clone, or to modify a cDNA clone to include at least one genomic intron. Methods for preparing cDNA and genomic clones are well known and within the level of ordinary skill in the art, and include the use of the sequence disclosed herein, or parts thereof, for probing or priming a library. Expression libraries can be probed with antibodies to zdint5 or other specific binding partners.

Zdint5 polynucleotide sequences disclosed herein can also be used as probes or primers to clone 5′ non-coding regions of a zdint5 gene. This gene is expected to provide for specific expression in colon, small intestine, fetal lung, testis, and B-cells. Promoter elements from a zdint5 gene could thus be used to direct the tissue-specific expression of heterologous genes in, for example, transgenic animals or patients treated with gene therapy. Cloning of 5′ flanking sequences also facilitates production of zdint5 proteins by “gene activation” as disclosed in U.S. Pat. No. 5,641,670. Briefly, expression of an endogenous zdint5 gene in a cell is altered by introducing into the zdint5 locus a DNA construct comprising at least a targeting sequence, a regulatory sequence, an exon, and an unpaired splice donor site. The targeting sequence is a zdint5 5′ non-coding sequence that permits homologous recombination of the construct with the endogenous zdint5 locus, whereby the sequences within the construct become operably linked with the endogenous zdint5 coding sequence. In this way, an endogenous zdint5 promoter can be replaced or supplemented with other regulatory sequences to provide enhanced, tissue-specific, or otherwise regulated expression.

The polynucleotides of the present invention can also be synthesized using DNA synthesizers. Currently the method of choice is the phosphoramidite method. If chemically synthesized double stranded DNA is required for an application such as the synthesis of a gene or a gene fragment, then each complementary strand is made separately. The production of short genes (60 to 80 bp) is technically straightforward and can be accomplished by synthesizing the complementary strands and then annealing them. For the production of longer genes (>300 bp), however, special strategies must be invoked, because the coupling efficiency of each cycle during chemical DNA synthesis is seldom 100%. To overcome this problem, synthetic genes (double-stranded) are assembled in modular form from single-stranded fragments that are from 20 to 100 nucleotides in length. See Glick and Pasternak, Molecular Biotechnology, Principles and Applications of Recombinant DNA, (ASM Press, Washington, D.C. 1994); Itakura et al., Annu. Rev. Biochem. 53: 323-356 (1984) and Climie et al., Proc. Natl. Acad. Sci. USA 87:633-7, 1990.

The present invention further provides counterpart polypeptides and polynucleotides from other species (orthologs). These species include, but are not limited to mammalian, avian, amphibian, reptile, fish, insect and other vertebrate and invertebrate species. Of particular interest are zdint5 polypeptides from other mammalian species, including murine, porcine, ovine, bovine, canine, feline, equine, and other primate polypeptides. Orthologs of human zdint5 can be cloned using information and compositions provided by the present invention in combination with conventional cloning techniques. For example, a cDNA can be cloned using mRNA obtained from a tissue or cell type that expresses zdint5 as disclosed herein. Such tissue would include, for example, colon, small intestine, fetal lung, testis, and B-cells. Suitable sources of mRNA can be identified by probing Northern blots with probes designed from the sequences disclosed herein. A library is then prepared from mRNA of a positive tissue or cell line. A zdint5-encoding cDNA can then be isolated by a variety of methods, such as by probing with a complete or partial human cDNA or with one or more sets of degenerate probes based on the disclosed sequences. A cDNA can also be cloned using the polymerase chain reaction, or PCR (Mullis, U.S. Pat. No. 4,683,202), using primers designed from the representative human zdint5 sequences disclosed herein. Within an additional method, the cDNA library can be used to transform or transfect host cells, and expression of the cDNA of interest can be detected with an antibody to zdint5 polypeptide. Similar techniques can also be applied to the isolation of genomic clones.

Those skilled in the art will recognize that the sequences disclosed in SEQ ID NOs:1, 3, 4, 6, 7, 9, 10 and 12 represent a single allele of human zdint5 and that allelic variation and alternative splicing are expected to occur. Allelic variants of this sequence can be cloned by probing cDNA or genomic libraries from different individuals according to standard procedures. Allelic variants of the DNA sequences shown in SEQ ID NOs: 1, 3, 4, 6, 7, 9, 10 and 12, including those containing silent mutations and those in which mutations result in amino acid sequence changes, are within the scope of the present invention, as are proteins which are allelic variants of SEQ ID NOs:2, 5, 8 and 11. cDNAs generated from alternatively spliced mRNAs, which retain the properties of the zdint5 polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

The present invention also provides isolated zdint5 polypeptides that are substantially similar to the polypeptides of SEQ ID NOs:2, 5, 8 and 11 and their orthologs. Such polypeptides will more preferably be at least 90% identical, and more preferably 95% or more identical to SEQ ID NOs:2, 5, 8 and 11 and their orthologs. Percent sequence identity is determined by conventional methods. See, for example, Altschul et al., Bull. Math. Bio. 48: 603-16, 1986 and Henikoff and Henikoff, Proc. Natl. Acad. Sci. USA 89:10915-9, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown in Table 3 (amino acids are indicated by the standard one-letter codes). The percent identity is then calculated as:

$$\frac{Total number of identical matches}{\begin{matrix} [ length of the longer sequence plus the \\ number of gaps introduced into the longer \\ sequence in order to align the two sequences ]\end{matrix}} \times 100$$

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Sequence identity of polynucleotide molecules is determined by similar methods using a ratio as disclosed above.

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant zdint5. The FASTA algorithm is described by Pearson and Lipman, Proc. Nat'l Acad. Sci. USA 85:2444 (1988), and by Pearson, Meth. Enzymol. 183:63 (1990).

Briefly, FASTA first characterizes sequence similarity by identifying regions shared by the query sequence (e.g., SEQ ID NOs:2, 5, 8 and 11) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from four to six.

The present invention includes nucleic acid molecules that encode a polypeptide having one or more conservative amino acid changes, compared with the amino acid sequences of SEQ ID NOs:2, 5, 8 and 11. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. As used herein, the language “conservative amino acid substitution” refers to a substitution represented by a BLOSUM62 value of greater than −1. For example, an amino acid substitution is conservative if the substitution is characterized by a BLOSUM62 value of 0, 1, 2, or 3. Preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Conservative amino acid changes in an zdint5 gene can be introduced by substituting nucleotides for the nucleotides recited in SEQ ID NOs: 1, 3, 4, 6, 7, 9, 10 and 12. Such “conservative amino acid” variants can be obtained, for example, by oligonucleotide-directed mutagenesis, linker-scanning mutagenesis, mutagenesis using the polymerase chain reaction, and the like (see Ausubel (1995) at pages 8-10 to 8-22; and McPherson (ed.), Directed Mutagenesis: A Practical Approach (IRL Press 1991)). The ability of such variants to promote cell-cell interactions can be determined using a standard method, such as the assay described herein. Alternatively, a variant zdint5 polypeptide can be identified by the ability to specifically bind anti-zdint5 antibodies.

Essential amino acids in the polypeptides of the present invention can be identified according to procedures known in the art, such as site-directed mutagenesis or alanine-scanning mutagenesis (Cunningham and Wells, Science 244: 1081-5, 1989; Bass et al., Proc. Natl. Acad. Sci. USA 88:4498-502, 1991). In the latter technique, single alanine mutations are introduced at every residue in the molecule, and the resultant mutant molecules are tested for biological activity as disclosed below to identify amino acid residues that are critical to the activity of the molecule. See also, Hilton et al., J. Biol. Chem. 271:4699-708, 1996. Sites of disintegrin-integrin, protease, or extracellular matrix interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related metalloprotease/disintegrin/thrombospondin-1 like molecules.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Variants of the disclosed zdint5 DNA and polypeptide sequences can be generated through DNA shuffling, as disclosed by Stemmer, Nature 370:389-91, 1994, Stemmer, Proc. Natl. Acad. Sci. USA 91:10747-51, 1994 and WIPO Publication WO 97/20078. Briefly, variant DNAs are generated by in vitro homologous recombination by random fragmentation of a parent DNA followed by reassembly using PCR, resulting in randomly introduced point mutations. This technique can be modified by using a family of parent DNAs, such as allelic variants or DNAs from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed herein can be combined with high-throughput, automated screening methods to detect activity of cloned, mutagenized polypeptides in host cells. Mutagenized DNA molecules that encode active polypeptides (e.g., protease activity, or angiogenesis inhibition) can be recovered from the host cells and rapidly sequenced using modem equipment. These methods allow the rapid determination of the importance of individual amino acid residues in a polypeptide of interest, and can be applied to polypeptides of unknown structure.

Regardless of the particular nucleotide sequence of a variant zdint5 gene, the gene encodes a polypeptide that is characterized by its protease activity, or angiogenesis inhibition, or by the ability to bind specifically to an anti-zdint5 antibody. More specifically, variant zdint5 genes encode polypeptides which exhibit at least 50%, and preferably, greater than 70, 80, or 90%, of the activity of polypeptide encoded by the human zdint5 gene described herein.

Variant zdint5 polypeptides or substantially homologous zdint5 polypeptides are characterized as having one or more amino acid substitutions, deletions or additions. These changes are preferably of a minor nature, that is conservative amino acid substitutions and other substitutions that do not significantly affect the folding or activity of the polypeptide; small deletions, typically of one to about 30 amino acids; and amino- or carboxyl-terminal extensions, such as an amino-terminal methionine residue, a small linker peptide of up to about 20-25 residues, or an affinity tag. The present invention thus includes polypeptides of from 18 to 2000 amino acid residues that comprise a sequence that is at least 85%, preferably at least 90%, and more preferably 95% or more identical to the corresponding region of SEQ ID NOs:2, 5, 8 or 11. Polypeptides comprising affinity tags can further comprise a proteolytic cleavage site between the zdint5 polypeptide and the affinity tag. Preferred such sites include thrombin cleavage sites and factor Xa cleavage sites.

For any zdint5 polypeptide, including variants and fusion proteins, one of ordinary skill in the art can readily generate a fully degenerate polynucleotide sequence encoding that variant using the information set forth in Tables 1 and 2 above. Moreover, those of skill in the art can use standard software to devise zdint5 variants based upon the nucleotide and amino acid sequences described herein. Accordingly, the present invention includes a computer-readable medium encoded with a data structure that provides at least one of the following sequences: SEQ ID NOs: 1, 2, 3, 4, 5, 6, 7, 8, 9, 10, 11, or 12. Suitable forms of computer-readable media include magnetic media and optically-readable media. Examples of magnetic media include a hard or fixed drive, a random access memory (RAM) chip, a floppy disk, digital linear tape (DLT), a disk cache, and a ZIP disk. Optically readable media are exemplified by compact discs (e.g., CD-read only memory (ROM), CD-rewritable (RW), and CD-recordable), and digital versatile/video discs (DVD) (e.g., DVD-ROM, DVD-RAM, and DVD+RW).

The present invention further provides a variety of other polypeptide fusions and related multimeric proteins comprising one or more polypeptide fusions. For example, a metalloprotease of TSP1-like polypeptide domain can be prepared as a fusion to a dimerizing protein, as disclosed in U.S. Pat. Nos. 5,155,027 and 5,567,584. Preferred dimerizing proteins in this regard include other disintegrin polypeptide domains, TSP1-like domains, disintegrin polypeptide domain fragments, or polypeptides comprising other members of the Disintegrin Protease family of proteins, such as, for example, members of the MDCs, SVMPs, METHs and ADAMs. These disintegrin polypeptide domain fusions, disintegrin polypeptide domain fragment fusions, or fusions with other Disintegrin Proteases can be expressed in genetically engineered cells to produce a variety of multimeric disintegrin-like analogs.

Fusion proteins can be prepared by methods known to those skilled in the art by preparing each component of the fusion protein and chemically conjugating them. Alternatively, a polynucleotide encoding both components of the fusion protein in the proper reading frame can be generated using known techniques and expressed by the methods described herein. For example, part or all of a domain(s) conferring a. biological function may be swapped between zdint5 of the present invention with the functionally equivalent domain(s) from another family member, such as ADAM, MDC, SVMP, ADAM-TS, and METH. Such domains include, but are not limited to, conserved motifs such as the secretory signal sequence, propeptide, protease, disintegrin and disintegrin loop domains, including the “RGD”-like sequence, the cysteine, transmembrane, TSP1-like and signaling domains. Such fusion proteins would be expected to have a biological functional profile that is the same or similar to polypeptides of the present invention or other known disintegrin-like family proteins (e.g. ADAMs, MDCs, SVMPs, ADAM-TS, and METH), depending on the fusion constructed. Moreover, such fusion proteins may exhibit other properties as disclosed herein.

Moreover, using methods described in the art, polypeptide fusions, or hybrid zdint5 proteins, are constructed using regions or domains of the inventive zdint5 in combination with those of other disintegrin and disintegrin-like molecules. (e.g. ADAM, MDC, and SVMP), or heterologous proteins (Sambrook et al., ibid., Altschul et al., ibid., Picard, Cur. Opin. Biology, 5:511-5, 1994, and references therein). These methods allow the determination of the biological importance of larger domains or regions in a polypeptide of interest. Such hybrids may alter reaction kinetics, binding, constrict or expand the substrate specificity, or alter tissue and cellular localization of a polypeptide, and can be applied to polypeptides of unknown structure.

Auxiliary domains can be fused to zdint5 polypeptides to target them to specific cells, tissues, or macromolecules (e.g., colon, small intestine, fetal lung, testis, and B-cells). For example, a protease polypeptide domain, or protease polypeptide fragment or protein, could be targeted to a predetermined cell type by fusing it to a disintegrin polypeptide domain or fragment that specifically binds to an integrin polypeptide or integrin-like polypeptide on the surface of the target cell. Such disintegrins or protease polypeptide domains or fragments can be fused to two or more moieties, such as an affinity tag for purification and a targeting-disintegrin domain. Similarly, a protease polypeptide domain, or protease polypeptide fragment or protein, could be targeted to the extracellular matrix by fusing it to a TSP1-like polypeptide domain or fragment that specifically binds to the extracellular matrix. In this way, polypeptides, polypeptide fragments and proteins can be targeted for therapeutic or diagnostic purposes. Polypeptide fusions can also comprise one or more cleavage sites., particularly between domains. See, Tuan et al., Connective Tissue Research 34:1-9, 1996.

Polypeptide fusions of the present invention will generally contain not more than about 2,000 amino acid residues, preferably not more than about 1,700 residues, more preferably not more than about 1,500 residues, and will in many cases be considerably smaller. For example, residues of zdint5 polypeptide can be fused to E. coli β-galactosidase (1,021 residues; see Casadaban et al., J. Bacteriol. 143:971-980, 1980), a 10-residue spacer, and a 4-residue factor Xa cleavage site. In a second example, residues of zdint5 polypeptide can be fused to maltose binding protein (approximately 370 residues), a 4-residue cleavage site, and a 6-residue polyhistidine tag.

To direct the export of a zdint5 polypeptide from the host cell, the zdint5 DNA is linked to a second DNA segment encoding a secretory peptide, such as a t-PA secretory peptide or a zdint5 secretory peptide. To facilitate purification of the secreted polypeptide, a C-terminal extension, such as a poly-histidine tag, substance P, Flag peptide (Hopp et al., Bio/Technology 6:1204-1210, 1988; available from Eastman Kodak Co., New Haven, Conn.), maltose binding protein, or another polypeptide or protein for which an antibody or other specific binding agent is available, can be fused to the zdint5 polypeptide.

The present invention also includes “functional fragments” of zdint5 polypeptides and nucleic acid molecules encoding such functional fragments. Routine deletion analyses of nucleic acid molecules can be performed to obtain functional fragments of a nucleic acid molecule that encodes an zdint5 polypeptide. As an illustration, DNA molecules having the nucleotide sequence of SEQ ID NOs:1, 3, 4, 6, 7, 9, 10 or 12 can be digested with Bal31 nuclease to obtain a series of nested deletions. The fragments are then inserted into expression vectors in proper reading frame, and the expressed polypeptides are isolated and tested for protease activity, or angiogenesis inhibition, or for the ability to bind anti-zdint5 antibodies. One alternative to exonuclease digestion is to use oligonucleotide-directed mutagenesis to introduce deletions or stop codons to specify production of a desired fragment. Alternatively, particular fragments of an zdint5 gene can be synthesized using the polymerase chain reaction.

Standard methods for identifying functional domains are well-known to those of skill in the art. For example, studies on the truncation at either or both termini of interferons have been summarized by Horisberger and Di Marco, Pharmac. Ther. 66:507 (1995). Moreover, standard techniques for functional analysis of proteins are described by, for example, Treuter et al., Molec. Gen. Genet. 240:113 (1993), Content et al., “Expression and preliminary deletion analysis of the 42 kDa 2-5A synthetase induced by human interferon,” in Biological Interferon Systems, Proceedings of ISIR-TNO Meeting on Interferon Systems, Cantell (ed.), pages 65-72 (Nijhoff 1987), Herschman, “The EGF Receptor,” in Control of Animal Cell Proliferation, Vol. 1, Boynton et al., (eds.) pages 169-199 (Academic Press 1985), Coumailleau et al., J. Biol. Chem. 270:29270 (1995); Fukunaga et al., J. Biol. Chem. 270:25291 (1995); Yamaguchi et al., Biochem. Pharmacol. 50:1295 (1995), and Meisel et al., Plant Molec. Biol. 30:1 (1996).

The present invention also contemplates functional fragments of an zdint5 gene that has amino acid changes, compared with the amino acid sequence of SEQ ID NOs:2, 5, 8 and 11. A variant zdint5 gene can be identified on the basis of structure by determining the level of identity with nucleotide and amino acid sequences of SEQ ID NOs: 1, 2, 3, 4, 5, 6, 7, 8 and 9, as discussed above. An alternative approach to identifying a variant gene on the basis of structure is to determine whether a nucleic acid molecule encoding a potential variant zdint5 gene can hybridize to a nucleic acid molecule having the nucleotide sequence of SEQ ID NOs:1, 2, 4, 6, 7, 9, 10 and 12, as discussed above.

Using the methods discussed herein, one of ordinary skill in the art can identify and/or prepare a variety of polypeptide fragments or variants of SEQ ID NOs:2, 5, 8, and 11 or that retain the metalloprotease, TSP1-like, and/or disintegrin activity of the wild-type zdint5 protein. Such polypeptides may include additional amino acids from, for example, a secretory domain, a propeptide domain, a protease domain, a disintegrin domain, a TSP1-like domain, a disintegrin loop (native or synthetic), part or all of a transmembrane and intracellular domains, including amino acids responsible for intracellular signaling; fusion domains; affinity tags; and the like.

The present invention also provides polypeptide fragments or peptides comprising an epitope-bearing portion of an zdint5 polypeptide described herein. Such fragments or peptides may comprise an “immunogenic epitope,” which is a part of a protein that elicits an antibody response when the entire protein is used as an immunogen. Immunogenic epitope-bearing peptides can be identified using standard methods (see, for example, Geysen et al., Proc. Nat'l Acad. Sci. USA 81:3998 (1983)).

In contrast, polypeptide fragments or peptides may comprise an “antigenic epitope,” which is a region of a protein molecule to which an antibody can specifically bind. Certain epitopes consist of a linear or contiguous stretch of amino acids, and the antigenicity of such an epitope is not disrupted by denaturing agents. It is known in the art that relatively short synthetic peptides that can mimic epitopes of a protein can be used to stimulate the production of antibodies against the protein (see, for example, Sutcliffe et al., Science 219:660 (1983)). Accordingly, antigenic epitope-bearing peptides and polypeptides of the present invention are useful to raise antibodies that bind with the polypeptides described herein. Antigenic epitope-bearing peptides include, for example, residues 727 to 732 of SEQ ID NO:11; residues 99 to 104 of SEQ ID NO:11; residues 726 to 731 of SEQ ID NO:11; and residues 127 to 132 of SEQ ID NO:11.

Antigenic epitope-bearing peptides and polypeptides contain at least four to ten amino acids, preferably at least ten to fifteen amino acids, more preferably 15 to 30 amino acids of SEQ ID NOs:2, 5, 8 and 11. Such epitope-bearing peptides and polypeptides can be produced by fragmenting a zdint5 polypeptide, or by chemical peptide synthesis, as described herein. Moreover, epitopes can be selected by phage display of random peptide libraries (see, for example, Lane and Stephen, Curr. Opin. Immunol. 5:268 (1993), and Cortese et al., Curr. Opin. Biotechnol. 7:616 (1996)). Standard methods for identifying epitopes and producing antibodies from small peptides that comprise an epitope are described, for example, by Mole, “Epitope Mapping,” in Methods in Molecular Biology, Vol. 10, Manson (ed.), pages 105-116 (The Humana Press, Inc. 1992), Price, “Production and Characterization of Synthetic Peptide-Derived Antibodies,” in Monoclonal Antibodies: Production, Engineering, and Clinical Application, Ritter and Ladyman (eds.), pages 60-84 (Cambridge University Press 1995), and Coligan et al. (eds.), Current Protocols in Immunology, pages 9.3.1-9.3.5 and pages 9.4.1-9.4.11 (John Wiley & Sons 1997).

As an illustration, potential antigenic sites in zdint5 (SEQ ID NOs: 2, 5, 8, and 11) were identified using the Jameson-Wolf method, Jameson and Wolf, CABIOS 4:181, (1988), as implemented by the PROTEAN program (version 3.14) of LASERGENE (DNASTAR; Madison, Wis.). Default parameters were used in this analysis.

Suitable antigens of zdint5 include: amino acid residues 23 to 29 as shown in SEQ ID NO:2; residues 40 to 49 as shown in SEQ ID NO:2; residues 62 to 70 as shown in SEQ ID NO:2; residues 87 to 98 as shown in SEQ ID NO:2; residues 108 to 120 as shown in SEQ ID NO:2; residues 137 to 144 as shown in SEQ ID NO:2; residues 157 to 172 as shown in SEQ ID NO:2; residues 177 to 183 as shown in SEQ ID NO:2; residues 190 to 197 as shown in SEQ ID NO:2; residues 23 to 49 as shown in SEQ ID NO:2; residues 40 to 70 as shown in SEQ ID NO:2; residues 62 to 98 as shown in SEQ ID NO:2; residues 87 to 120 as shown in SEQ ID NO:2; residues 108 to 144 as shown in SEQ ID NO:2 ; residues 137 to 172 as shown in SEQ ID NO:2 ; residues 157 to 183 as shown in SEQ ID NO:2; residues 177 to 197 as shown in SEQ ID NO:2; residues 3 to 24 as shown in SEQ ID NO:5; residues 39 to 45 as shown in SEQ ID NO:5; residues 3 to 45 as shown in SEQ ID NO:5; residues 31 to 18 as shown in SEQ ID NO:5; residues 26 to 37 as shown in SEQ ID NO:8; residues 39 to 59 as shown in SEQ ID NO:8; residues 21 to 37 as shown in SEQ ID NO:8; residues 26 to 59 as shown in SEQ ID NO:8; residues 12 to 20 as shown in SEQ ID NO:8; residues 31 to 42 as shown in SEQ ID NO:1; residues 60 to 67 as shown in SEQ ID NO:11; residues 81 to 108 as shown in SEQ ID NO:11; residues 126 to 132 as shown in SEQ ID NO:11; residues 143 to 152 as shown in SEQ ID NO:11; residues 165 to 173 as shown in SEQ ID NO:1; residues 190 to 203 as shown in SEQ ID NO:11; residues 211 to 223 as shown in SEQ ID NO:1; residues 240 to 247 as shown in SEQ ID NO:1; residues 260 to 275 as shown in SEQ ID NO:1; residues 280 to 287 as shown in SEQ ID NO:11; residues 293 to 300 as shown in SEQ ID NO:1; residues 310 to 327 as shown in SEQ ID NO:11; residues 351 to 359 as shown in SEQ ID NO:11; residues 366 to 375 as shown in SEQ ID NO:11; residues 381 to 419 as shown in SEQ ID NO:1; residues 434 to 463 as shown in SEQ ID NO:11; residues 456 to 463 as shown in SEQ ID NO:11; residues 469 to 499 as shown in SEQ ID NO:1; residues 505 to 527 as shown in SEQ ID NO:11; residues 529 to 551 as shown in SEQ ID NO:11; residues 591 to 598 as shown in SEQ ID NO:11; residues 601 to 621 as shown in SEQ ID NO:11; residues 633 to 639 as shown in SEQ ID NO:11; residues 641 to 657 as shown in SEQ ID NO:11; residues 671 to 690 as shown in SEQ ID NO:11; residues 709 to 715 as shown in SEQ ID NO:11; residues 724 to 735 as shown in SEQ ID NO:11; residues 738 to 761 as shown in SEQ ID NO:11; residues 775 to 781 as shown in SEQ ID NO:11; residues 785 to 793 as shown in SEQ ID NO:11; residues 12 to 42 as shown in SEQ ID NO:11; residues 31 to 67 as shown in SEQ ID NO:2; residues 60 to 108 as shown in SEQ ID NO:2; residues 81 to 132 as shown in SEQ ID NO:2; residues 126 to 152 as shown in SEQ ID NO:2; residues 143 to 173 as shown in SEQ ID NO:2; residues 165 to 203 as shown in SEQ ID NO:2; residues 190 to 223 as shown in SEQ ID NO:2; residues 211 to 247 as shown in SEQ ID NO:2; residues 240 to 275 as shown in SEQ ID NO:2; residues 260 to 287 as shown in SEQ ID NO:2; residues 280 to 300 as shown in SEQ ID NO:2; residues 293 to 327 as shown in SEQ ID NO:2; residues 310 to 359 as shown in SEQ ID NO:2; residues 351 to 375 as shown in SEQ ID NO:2; residues 366 to 419 as shown in SEQ ID NO:2; residues 381 to 463 as shown in SEQ ID NO:2; residues 456 to 527 as shown in SEQ ID NO:2; residues 505 to 551 as shown in SEQ ID NO:2; residues 529 to 621 as shown in SEQ ID NO:2; residues 601 to 639 as shown in SEQ ID NO:2; residues 633 to 657 as shown in SEQ ID NO:2; residues 641 to 690 as shown in SEQ ID NO:2; residues 671 to 715 as shown in SEQ ID NO:2; residues 709 to 735 as shown in SEQ ID NO:2; residues 724 to 761 as shown in SEQ ID NO:2; residues 738 to 781 as shown in SEQ ID NO:2; and residues 775 to 793 as shown in SEQ ID NO:2 are antigenic peptides.

Zdint5 polypeptides can also be used to prepare antibodies that specifically bind to zdint5 epitopes, peptides or polypeptides. The zdint5 polypeptide or a fragment thereof serves as an antigen (immunogen) to inoculate an animal and elicit an immune response. One of skill in the art would recognize that antigenic, epitope-bearing polypeptides contain a sequence of at least 6, preferably at least 9, and more preferably at least 15 to about 30 contiguous amino acid residues of a zdint5 polypeptide (e.g., SEQ ID NOs:2, 5, 8 and 11). Polypeptides comprising a larger portion of a zdint5 polypeptide, i.e., from 30 to 10 residues up to the entire length of the amino acid sequence are included. Antigens or immunogenic epitopes can also include attached tags, adjuvants and carriers, as described herein. Suitable antigens include the zdint5 polypeptides encoded by SEQ ID NOs:2, 5, 8 or 11 from amino acid number 1 to amino acid number 1120, or a contiguous 9 to 1170 amino acid fragment thereof. Preferred peptides to use as antigens are hydrophilic peptides such as those predicted by one of skill in the art from a hydrophobicity plot. zdint5 hydrophilic peptides include peptides comprising amino acid sequences selected from the group consisting of: residues 20 to 32 of SEQ ID NO:2; residues 64 to 69 of SEQ ID NO:2; residues 86 to 98 of SEQ ID NO:2; residues 110 to 121 of SEQ ID NO:2; residues 154 to 171 of SEQ ID NO:2; residues 190 to 199 of SEQ ID NO:2; residues 20 to 69 of SEQ ID NO:2; residues 64 to 98 of SEQ ID NO:2; residues 86 to 121 of SEQ ID NO:2; residues 110 to 171 of SEQ ID NO:2; residues 154 to 199 of SEQ ID NO:2; residues 1 to 13 of SEQ ID NO:5; residues 15 to 31 of SEQ ID NO:5; residues 1 to 31 of SEQ ID NO:5; residues 25 to 36 of SEQ ID NO:8; residues 41 to 54 of SEQ ID NO:8; residues 25 to 54 of SEQ ID NO:8; residues 13 to 19 of SEQ ID NO:11; residues 30 to 40 of SEQ ID NO:11; residues 60 to 66 of SEQ ID NO:11; residues 85 to 107 of SEQ ID NO:11; residues 123 to 135 of SEQ ID NO:11; residues 167 to 172 of SEQ ID NO:11; residues 189 to 201 of SEQ ID NO:11; residues 213 to 225 of SEQ ID NO:11; residues 257 to 274 of SEQ ID NO:11; residues 293 to 302 of SEQ ID NO:11; residues 309 to 327 of SEQ ID NO:11; residues 334 to 342 of SEQ ID NO:11; residues 348 to 358 of SEQ ID NO:11; residues 366 to 374 of SEQ ID NO:11; residues 386 to 408 of SEQ ID NO:11; residues 410 to 425 of SEQ ID NO:11; residues 472 to 499 of SEQ ID NO:11; residues 509 to 525 of SEQ ID NO:11; residues 527 to 551 of SEQ ID NO:11; residues 591 to 619 of SEQ ID NO:11; residues 621 to 626 of SEQ ID NO:11; residues 635 to 649 of SEQ ID NO:11; residues 682 to 690 of SEQ ID NO:11; residues 695 to 702 of SEQ ID NO:11; residues 723 to 734 of SEQ ID NO:11; residues 739 to 752 of SEQ ID NO:11; residues 755 to 763 of SEQ ID NO:11; and residues 786 to 793 of SEQ ID NO:11. Additionally, antigens can be generated to portions of the polypeptide which are likely to be on the surface of the folded protein. These antigens include: residues 22 to 31 of SEQ ID NO:2; residues 87 to 97 of SEQ ID NO:2; residues 113 to 118 of SEQ ID NO:2; residues 26 to 32 of SEQ ID NO:8; residues 44 to 50 of SEQ ID NO:8; residues 31 to 39 of SEQ ID NO:11; residues 86 to 104 of SEQ ID NO:11; residues 125 to 134 of SEQ ID NO:11; residues 190 to 200 of residues 216 to 221 of SEQ ID NO:11; SEQ ID NO:11; residues 492 to 498 of SEQ ID residues 516 to 522 of SEQ ID NO:11; NO:11; residues 546 to 551 of SEQ ID NO:11; residues 593 to 598 of SEQ ID NO:11; residues 724 to 730 of SEQ ID NO:11; residues 742 to 748 of SEQ ID NO:11; residues 787 to 793 of SEQ ID NO:11; residues 13 to 40 of SEQ ID NO:11; residues 30 to 66 of SEQ ID NO:11; residues 60 to 107 of SEQ ID NO:11; residues 85 to 135 of SEQ ID NO:11; residues 123 to 172 of SEQ ID NO:11; residues 167 to 201 of SEQ ID NO:11; residues 189 to 225 of SEQ ID NO:11; residues 213 to 274 of SEQ ID NO:11; residues 257 to 302 of SEQ ID NO:11; residues 293 to 327 of SEQ ID NO:11; residues 309 to 342 of SEQ ID NO:11; residues 334 to 358 of SEQ ID NO:11; residues 348 to 376 of SEQ ID NO:11; residues 366 to 408 of SEQ ID NO:11; residues 386 to 425 of SEQ ID NO:11; residues 410 to 499 of SEQ ID NO:11; residues 472 to 525 of SEQ ID NO:11; residues 509 to 551 of SEQ ID NO:11; residues 527 to 619 of SEQ ID NO:11; residues 621 to 649 of SEQ ID NO:11; residues 635 to 690 of SEQ ID NO:11; residues 682 to 702 of SEQ ID NO:11; residues 695 to 734 of SEQ ID NO:11; residues 723 to 752 of SEQ ID NO:11; residues 739 to 763 of SEQ ID NO:11; and residues 755 to 793 of SEQ ID NO:11. Antibodies from an immune response generated by inoculation of an animal with these antigens can be isolated and purified as described herein. Methods for preparing and isolating polyclonal and monoclonal antibodies are well known in the art. See, for example, Current Protocols in Immunology, Cooligan, et al. (eds.), National Institutes of Health, John Wiley and Sons, Inc., 1995; Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982.

As would be evident to one of ordinary skill in the art, polyclonal antibodies can be generated from inoculating a variety of warm-blooded animals such as horses, cows, goats, sheep, dogs, chickens, rabbits, mice, and rats with a zdint5 polypeptide or a fragment thereof. The immunogenicity of a zdint5 polypeptide may be increased through the use of an adjuvant, such as alum (aluminum hydroxide) or Freund's complete or incomplete adjuvant. Polypeptides useful for immunization also include fusion polypeptides, such as fusions of zdint5 or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

As used herein, the term “antibodies” includes polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced.

Alternative techniques for generating or selecting antibodies useful herein include in vitro exposure of lymphocytes to zdint5 protein or peptide, and selection of antibody display libraries in phage or similar vectors (for instance, through use of immobilized or labeled zdint5 protein or peptide). Genes encoding polypeptides having potential zdint5 polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides which interact with a known target which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially, for instance from CLONTECH Laboratories, Inc., (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Random peptide display libraries can be screened using the zdint5 sequences disclosed herein to identify proteins which bind to zdint5. These “binding proteins” which interact with zdint5 polypeptides can be used for tagging cells; for isolating homolog polypeptides by affinity purification; they can be directly or indirectly conjugated to drugs, toxins, radionuclides and the like. These binding proteins can also be used in analytical methods such as for screening expression libraries and neutralizing activity. The binding proteins can also be used for diagnostic assays for determining circulating levels of polypeptides; for detecting or quantitating soluble polypeptides as marker of underlying pathology or disease. These binding proteins can also act as zdint5 “antagonists” to block zdint5 binding and signal transduction in vitro and in vivo. These anti-zdint5 binding proteins would be useful for modulating, for example, protease activity, or angiogenesis inhibition, in general.

Antibodies are determined to be specifically binding if they exhibit a threshold level of binding activity (to a zdint5 polypeptide, peptide or epitope) of at least 10-fold greater than the binding affinity to a control (non-zdint5) polypeptide. The binding affinity of an antibody can be readily determined by one of ordinary skill in the art, for example, by Scatchard analysis (Scatchard, G., Ann. NY Acad. Sci. 51: 660-672, 1949).

A variety of assays known to those skilled in the art can be utilized to detect antibodies which specifically bind to zdint5 proteins or peptides. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmunoprecipitation, enzyme-linked immunosorbent assay (ELISA), dot blot or Western blot assay, inhibition or competition assay, and sandwich assay. In addition, antibodies can be screened for binding to wild-type versus mutant zdint5 protein or polypeptide.

Antibodies to zdint5 may be used for tagging cells that express zdint5; for isolating zdint5 by affinity purification; for diagnostic assays for determining circulating levels of zdint5 polypeptides; for detecting or quantitating soluble zdint5 as marker of underlying pathology or disease; in analytical methods employing FACS; for screening expression libraries; for generating anti-idiotypic antibodies; and as neutralizing antibodies or as antagonists to block zdint5 in vitro and in vivo. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Antibodies herein may also be directly or indirectly conjugated to drugs, toxins, radionuclides and the like, and these conjugates used for in vivo diagnostic or therapeutic applications. Moreover, antibodies to zdint5 or fragments thereof may be used in vitro to detect denatured zdint5 or fragments thereof in assays, for example, Western Blots or other assays known in the art.

Antibodies or polypeptides herein can also be directly or indirectly conjugated to drugs, toxins, radionuclides and the like, and these conjugates used for in vivo diagnostic or therapeutic applications. For instance, polypeptides or antibodies of the present invention can be used to identify or treat tissues or organs that express a corresponding anti-complementary molecule (integrin or antigen, respectively, for instance). More specifically, zdint5 polypeptides or anti-zdint5 antibodies, or bioactive fragments or portions thereof, can be coupled to detectable or cytotoxic molecules and delivered to a mammal having cells, tissues or organs that express the anti-complementary molecule.

Suitable detectable molecules may be directly or indirectly attached to the polypeptide or antibody, and include radionuclides, enzymes, substrates, cofactors, inhibitors, fluorescent markers, chemiluminescent markers, magnetic particles and the like. Suitable cytotoxic molecules may be directly or indirectly attached to the polypeptide or antibody, and include bacterial or plant toxins (for instance, diphtheria toxin, Pseudomonas exotoxin, ricin, abrin and the like), as well as therapeutic radionuclides, such as iodine-131, rhenium-188 or yttrium-90 (either directly attached to the polypeptide or antibody, or indirectly attached through means of a chelating moiety, for instance). Polypeptides or antibodies may also be conjugated to cytotoxic drugs, such as adriamycin. For indirect attachment of a detectable or cytotoxic molecule, the detectable or cytotoxic molecule can be conjugated with a member of a complementary/anticomplementary pair, where the other member is bound to the polypeptide or antibody portion. For these purposes, biotin/streptavidin is an exemplary complementary/anticomplementary pair.

In another embodiment, polypeptide-toxin fusion proteins or antibody-toxin fusion proteins can be used for targeted cell or tissue inhibition or ablation (for instance, to treat cancer cells or tissues). Alternatively, a fusion protein including only the TSP1-like domain may be suitable for directing a detectable molecule, a cytotoxic molecule or a complementary molecule to a cell or tissue type of interest (i.e., extracellular matrix). Similarly, the corresponding binding partner to zdint5 can be conjugated to a detectable or cytotoxic molecule and provide a generic targeting vehicle for cell/tissue-specific delivery of generic anti-complementary-detectable/cytotoxic molecule conjugates.

In another embodiment, zdint5-cytokine fusion proteins or antibody-cytokine fusion proteins can be used for enhancing in vivo killing of target tissues (for example, colon, small intestine, fetal lung, testis, and B-cells), if the zdint5 polypeptide or anti-zdint5 antibody targets hyperproliferative tissues from these organs. (See, generally, Hornick et al., Blood 89:4437-47, 1997). They described fusion proteins that enable targeting of a cytokine to a desired site of action, thereby providing an elevated local concentration of cytokine. Suitable zdint5 polypeptides or anti-zdint5 antibodies target an undesirable cell or tissue (i.e., a tumor or a leukemia), and the fused cytokine mediates improved target cell lysis by effector cells. Suitable cytokines for this purpose include interleukin 2 and granulocyte-macrophage colony-stimulating factor (GM-CSF), for instance.

The bioactive polypeptide or antibody conjugates described herein can be delivered intravenously, intraarterially or intraductally, or may be introduced locally at the intended site of action.

The zdint5 polypeptides of the present invention, including full-length polypeptides, biologically active fragments, and fusion polypeptides, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., NY, 1987.

In general, a DNA sequence encoding a zdint5 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a zdint5 polypeptide into the secretory pathway of a host cell, a secretory signal sequence (also known as a leader sequence, prepro sequence or pre sequence) is provided in the expression vector. The secretory signal sequence may be that of zdint5, or may be derived from another secreted protein (e.g., t-PA) or synthesized de novo. The secretory signal sequence is operably linked to the zdint5 DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain secretory signal sequences may be positioned elsewhere in the DNA sequence of interest (see, e.g., Welch et al., U.S. Pat. No. 5,037,743; Holland et al., U.S. Pat. No. 5,143,830).

The native secretory signal sequence of the polypeptides of the present invention is used to direct other polypeptides into the secretory pathway. The present invention provides for such fusion polypeptides. A signal fusion polypeptide can be made wherein a secretory signal sequence derived from a zdint5 polypeptide is operably linked to another polypeptide using methods known in the art and disclosed herein. The secretory signal sequence contained in the fusion polypeptides of the present invention is preferably fused amino-terminally to an additional peptide to direct the additional peptide into the secretory pathway. Such constructs have numerous applications known in the art. For example, these novel secretory signal sequence fusion constructs can direct the secretion of an active component of a normally non-secreted protein, such as a receptor. Such fusions may be used in vivo or in vitro to direct peptides through the secretory pathway.

Alternatively, the protease domain of zdint5 can be substituted by a heterologous sequence providing a different protease domain. In this case, the fusion product can be secreted, and the TSP1-like domain of zdint5 can direct the substituted protease domain to a specific tissue described above. This substituted protease domain can be chosen from the protease domains represented by the ADAMs/MDCs/SVMPs/ADAM-TS/METH like family members, or domains from other known proteases. Similarly, the TSP1-like domain of zdint5 protein can be substituted by a heterlogous sequence providing a different TSP1-like domain. Again, the fusion product can be secreted and the substituted TSP1-like domain can target the protease domain of zdint5 to a specific tissue. The substituted TSP1-like domain can be chosen from the TSP1-like domains of the ADAMs/MDCs/SVMPs/METH-like family members. In these cases, the fusion products can be soluble or membrane-anchored proteins.

Cultured mammalian cells are suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-5, 1982), DEAE-dextran mediated transfection (Ausubel et al., ibid.), and liposome-mediated transfection (Hawley-Nelson et al., Focus 15:73, 1993; Ciccarone et al., Focus 15:80, 1993, and viral vectors (Miller and Rosman, BioTechniques 7:980-90, 1989; Wang and Finer, Nature Med. 2:714-6, 1996). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL, 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection (Manasas, Va.). In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g., hygromycin resistance, multi-drug resistance, puromycin acetyltransferase) can also be used. Alternative markers that introduce an altered phenotype, such as green fluorescent protein, or cell surface proteins, such as CD4, CD8, Class I MHC, or placental alkaline phosphatase, may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Bangalore), 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). See, King, L. A. and Possee, R. D., The Baculovirus Expression System: A Laboratory Guide, London, Chapman & Hall; O'Reilly, D. R. et al., Baculovirus Expression Vectors: A Laboratory Manual, New York, Oxford University Press., 1994; and, Richardson, C. D., Ed., Baculovirus Expression Protocols. Methods in Molecular Biology, Totowa, N.J., Humana Press, 1995. A second method of making recombinant zdint5 baculovirus utilizes a transposon-based system described by Luckow (Luckow, V. A, et al., J Virol 67:4566-79, 1993). This system, which utilizes transfer vectors, is sold in the Bac-to-Bac™ kit (Life Technologies, Rockville, Md.). This system utilizes a transfer vector, pFastBac1™ (Life Technologies) containing a Tn7 transposon to move the DNA encoding the zdint5 polypeptide into a baculovirus genome maintained in E. coli as a large plasmid called a “bacmid.” The pFastBac1™ transfer vector utilizes the AcNPV polyhedrin promoter to drive the expression of the gene of interest, in this case zdint5. However, pFastBac1™ can be modified to a considerable degree. The polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins. See, Hill-Perkins, M. S. and Possee, R. D., J. Gen. Virol. 71:971-6, 1990; Bonning, B. C. et al., J. Gen. Virol. 75:1551-6, 1994; and, Chazenbalk, G. D., and Rapoport, B., J. Biol Chem 270:1543-9, 1995. In such transfer vector constructs, a short or long version of the basic protein promoter can be used. Moreover, transfer vectors can be constructed which replace the native zdint5 secretory signal sequences with secretory signal sequences derived from insect proteins. For example, a secretory signal sequence from Ecdysteroid Glucosyltransferase (EGT), honey bee Melittin (Invitrogen, Carlsbad, Calif.), or baculovirus gp67 (PharMingen, San Diego, Calif.) can be used in constructs to replace the native zdint5 secretory signal sequence. In addition, transfer vectors can include an in-frame fusion with DNA encoding an epitope tag at the C- or N-terminus of the expressed zdint5 polypeptide, for example, a Glu-Glu epitope tag (Grussenmeyer, T. et al., Proc. Natl. Acad. Sci. 82:7952-4, 1985). Using a technique known in the art, a transfer vector containing zdint5 is transformed into E. coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses zdint5 is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO™ cell line (Invitrogen) derived from Trichoplusia ni (U.S. Pat. No. 5,300,435). Commercially available serum-free media are used to grow and maintain the cells. Suitable media are Sf900 II™ (Life Technologies) or ESF 921™ (Expression Systems) for the Sf9 cells; and Ex-cellO405™ (JRH Biosciences, Lenexa, Kans.) or Express FiveO™ (Life Technologies) for the T. ni cells. The cells are grown up from an inoculation density of approximately 2-5×10⁵ cells to a density of 1-2×10⁶ cells at which time a recombinant viral stock is added at a multiplicity of infection (MOI) of 0.1 to 10, more typically near 3. Procedures used are generally described in available laboratory manuals (King, L. A. and Possee, R. D., ibid.; O'Reilly, D. R. et al., ibid.; Richardson, C. D., ibid.). Subsequent purification of the zdint5 polypeptide from the supernatant can be achieved using methods described herein.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillernondii and Candida maltosa are known in the art. See, for example, Gleeson et al., J. Gen. Microbiol. 132:3459-65, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al, U.S. Pat. No. 5,162,228. Methods for transforming Neurospora are disclosed by Lambowitz, U.S. Pat. No. 4,486,533. The use of Pichia methanolica as host for the production of recombinant proteins is disclosed in U.S. Pat. Nos. 5,716,808, 5,736,383, 5,854,039, and 5,888,768.

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a zdint5 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell. P. methanolica cells are cultured in a medium comprising adequate sources of carbon, nitrogen and trace nutrients at a temperature of about 25° C. to 35° C. Liquid cultures are provided with sufficient aeration by conventional means, such as shaking of small flasks or sparging of fermentors. A preferred culture medium for P. methanolica is YEPD (2% D-glucose, 2% Bacto™ Peptone (Difco Laboratories, Detroit, Mich.), 1% Bacto™ yeast extract (Difco Laboratories), 0.004% adenine and 0.006% L-leucine).

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for zdint5 amino acid residues.

It is preferred to purify the polypeptides of the present invention to ≧80% purity, more preferably to ≧90% purity, even more preferably ≧95% purity, and particularly preferred is a pharmaceutically pure state, that is greater than 99.9% pure with respect to contaminating macromolecules, particularly other proteins and nucleic acids, and free of infectious and pyrogenic agents. Preferably, a purified polypeptide is substantially free of other polypeptides, particularly other polypeptides of animal origin.

Expressed recombinant zdint5 proteins (including chimeric polypeptides and multimeric proteins) are purified by conventional protein purification methods, typically by a combination of chromatographic techniques. See, in general, Affinity Chromatography: Principles & Methods, Pharmacia LKB Biotechnology, Uppsala, Sweden, 1988; and Scopes, Protein Purification: Principles and Practice, Springer-Verlag, New York, 1994. Proteins comprising a polyhistidine affinity tag (typically about 6 histidine residues) are purified by affinity chromatography on a nickel chelate resin. See, for example, Houchuli et al., Bio/Technol. 6: 1321-1325, 1988. Proteins comprising a Glu-Glu tag can be purified by immunoaffinity chromatography according to conventional procedures. See, for example, Grussenmeyer et al., ibid. Maltose binding protein fusions are purified on an amylose column according to methods known in the art.

The polypeptides of the present invention can be isolated by a combination of procedures including, but not limited to, anion and cation exchange chromatography, size exclusion, and affinity chromatography. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, M. Deutscher, (ed.), Acad. Press, San Diego, 1990, pp.529-39). Within additional embodiments of the invention, a fusion of the polypeptide of interest and an affinity tag (e.g., maltose-binding protein, an immunoglobulin domain) may be constructed to facilitate purification.

Zdint5 polypeptides can also be prepared through chemical synthesis according to methods known in the art, including exclusive solid phase synthesis, partial solid phase methods, fragment condensation or classical solution synthesis. See, for example, Merrifield, J. Am. Chem. Soc. 85:2149, 1963; Stewart et al., Solid Phase Peptide Synthesis (2nd edition), Pierce Chemical Co., Rockford, Ill., 1984; Bayer and Rapp, Chem. Pept. Prot. 3:3, 1986; and Atherton et al., Solid Phase Peptide Synthesis: A Practical Approach, IRL Press, Oxford, 1989. In vitro synthesis is particularly advantageous for the preparation of smaller polypeptides.

Using methods known in the art, zdint5 proteins can be prepared as monomers or multimers; glycosylated or non-glycosylated; pegylated or non-pegylated; and may or may not include an initial methionine amino acid residue.

The metalloprotease (SEQ ID NO:2) and TSP1-like domains (i.e., SEQ ID NOs:5 and 8) are of particular interest for use in assays and treatment of disorders of the colon, small intestine, fetal lung, testis, and B-cells. The metalloprotease domain may be involved in activating the host defense system against infection. One such metalloprotease is matrilysin (Wilson, C. et al., Science 286: 113-117, 1999). Matrilysin has been shown to be involved in the host defense to bacterial pathogens in the small intestine. Wilson et al. also suggest that as an epithelial-associated protein, matrilysin may specifically regulate defensin activation in other tissue than the small intestine. Similarly, the zdint5 metalloprotease domain (SEQ ID NO:2) alone, or in conjunction with other domains (i.e., the TSP1-like domains of zdint5, SEQ ID NOs: 5 and 8, or SEQ ID NO:11, ot other TSP1-like domains from the ADAM-TS and METH prtoein families) may be involved in the body's response to pathogenic bacterial invading the epithelium of colon, small intestine, and lung, for example.

Additionally, as a protease the metalloprotease domain (SEQ ID NO:2) can be used as an enzymatic detergent for use in industrial applications. One skilled in the art can readily identify assays to measure the proteolytic function of zdint5 molecules.

An exemplary assay to measure the activity of the metalloprotease domain may be by measuring its ability to bind a plasma proteolytic enzyme inhibitor, α2M. This protein contains a bait region that provides a target for the proteases including metalloproteases. Cleavage of the bait region triggers conformational changes in the α2M subunits that cause an encapsulation of the protease and activation of the internal thioesters of α2M, resulting in a covalent cross-linking of the active protease. See also, Nagase, H. et al., Ann. N. Y. Acad. Sci. 732: 294-302, 1994.

Heparin and heparin sulfate are molecules which facilitate the binding of secreted growth factors and other proteins to the extracellular matrix. Thus molecules which bind heparin and heparin sulfate are useful to modulate the effects of these growth factors, etc. Heparin and heparin binding motifs have been identified in thrombospondin repeats in the METH and ADAM-TS subgroups of proteins (see Kuno, 1998, ibid). Thus, the TSP1-like domains (SEQ ID NOs: 5 and/or 8) may be useful in modulating the effects of growth factors by binding to heparin and heparin sulfate.

The activity of zdint5 polypeptides can be measured using a variety of assays that measure, for example, protease activity, angiogenesis inhibition; extracellular matrix formation or remodeling; metastasis, and other biological functions associated with ADAM/MDC/SVMP/ADAM-TS/METH family members or with integrin/disintegrin interactions, such as, apoptosis; or differentiation, for example. Of particular interest is a change in tumor suppression.

Proteins, including alternatively spliced peptides, of the present invention are useful for tumor suppression, gamete maturation, immunologic recognition, and growth and differentiation either working in isolation, or in conjunction with other molecules (growth factors, cytokines, etc.) in colon, small intestine, fetal lung, testis, and B-cells. Alternative splicing of zdint5 may cell-type specific and confer activity to specific tissues.

Another assay of interest measures or detects changes in proliferation, differentiation, and/or development of intestinal, colon or lung tissues. Additionally, the effects of zdint5 polypeptides on protease activity, or angiogenesis inhibition of endothelial cells, in general, and tumor cells would be of interest to measure. Yet other assays examine changes in protease activity and apoptosis.

Proliferation can be measured using cultured cells or in vivo by administering molecules of the claimed invention to an appropriate animal model. Generally, proliferative effects are observed as an increase in cell number and therefore, may include inhibition of apoptosis, as well as mitogenesis. Cultured cells include colon adenocarcinoma, testis, fetal and adult lung, B cells, melanoma and human umbilical vein endothelial cells from primary cultures. Assays measuring cell proliferation are well known in the art. For example, assays measuring proliferation include such assays as chemosensitivity to neutral red dye (Cavanaugh et al., Investigational New Drugs 8:347-354, 1990), incorporation of radiolabelled nucleotides (Cook et al., Analytical Biochem. 179:1-7, 1989), incorporation of 5-bromo-2′-deoxyuridine (BrdU) in the DNA of proliferating cells (Porstmann et al., J. Immunol. Methods 82:169-179, 1985), and use of tetrazolium salts (Mosmann, J. Immunol. Methods 65:55-63, 1983; Alley et al., Cancer Res. 48:589-601, 1988; Marshall et al., Growth Reg. 5:69-84, 1995; and Scudiero et al., Cancer Res. 48:4827-4833, 1988). Additionally, zdint5 polypeptides may play a role cell proliferation, migration, and angiogenesis by mediating cell adhesion.

To determine if zdint5 is a chemotractant in vivo, zdint5 can be given by intradermal or intraperitoneal injection. Characterization of the accumulated leukocytes at the site of injection can be determined using lineage specific cell surface markers and fluorescence immunocytometry or by immunohistochemistry (Jose, J. Exp. Med. 179:881-87, 1994). Release of specific leukocyte cell populations from bone marrow into peripheral blood can also be measured after zdint5 injection.

Differentiation is a progressive and dynamic process, beginning with pluripotent stem cells and ending with terminally differentiated cells. Pluripotent stem cells that can regenerate without commitment to a lineage express a set of differentiation markers that are lost when commitment to a cell lineage is made. Progenitor cells express a set of differentiation markers that may or may not continue to be expressed as the cells progress down the cell lineage pathway toward maturation. Differentiation markers that are expressed exclusively by mature cells are usually functional properties such as cell products, enzymes to produce cell products and receptors and receptor-like complementary molecules. The stage of a cell population's differentiation is monitored by identification of markers present in the cell population. For example, myocytes, osteoblasts, adipocytes, chrondrocytes, fibroblasts and reticular cells are believed to originate from a common mesenchymal stem cell (Owen et al., Ciba Fdn. Symp. 136:42-46, 1988). Markers for mesenchymal stem cells have not been well identified (Owen et al., J. of Cell Sci. 87:731-738, 1987), so identification is usually made at the progenitor and mature cell stages.

There is evidence to suggest that factors that stimulate specific cell types down a pathway towards terminal differentiation or dedifferentiation affect the entire cell population originating from a common precursor or stem cell. Thus, zdint5 polypeptides may stimulate inhibition or proliferation of endocrine and exocrine cells of the colon, small intestine, fetal lung, testis, and B-cells.

Assays measuring differentiation include, for example, measuring cell-surface markers associated with stage-specific expression of a tissue, enzymatic activity, functional activity or morphological changes (Watt, FASEB, 5:281-284, 1991; Francis, Differentiation 57:63-75, 1994; Raes, Adv. Anim. Cell Biol. Technol. Bioprocesses, 161-171, 1989).0

The zdint5 polypeptides of the present invention can be used to study proliferation or differentiation in colon, small intestine, fetal lung, testis, and B-cells. Such methods of the present invention generally comprise incubating cells derived from these tissues in the presence and absence of zdint5 polypeptide, monoclonal antibody, agonist or antagonist thereof and observing changes in cell proliferation or differentiation. Cell lines from these tissues are commercially available from, for example, American Type Culture Collection (Manasas, Va.).

Proteins, including alternatively spliced peptides, and fragments, of the present invention are useful for studying protease activity, or angiogenesis inhibition, zdint5 molecules, variants, and fragments can be applied in isolation, or in conjunction with other molecules (growth factors, cytokines, etc.) in colon, small intestine, fetal lung, testis, and B-cells.

Proteins of the present invention are useful for delivery of therapeutic agents such as, but not limited to, proteases, radionuclides, chemotherapy agents, and small molecules. Effects of these therapeutic agents can be measured in vitro using cultured cells, ex vivo on tissue slices, or in vivo by administering molecules of the claimed invention to the appropriate animal model. An alternative in vivo approach for assaying proteins of the present invention involves viral delivery systems. Exemplary viruses for this purpose include adenovirus, herpesvirus, lentivirus, vaccinia virus and adeno-associated virus (AAV). Adenovirus, a double-stranded DNA virus, is currently the best studied gene transfer vector for delivery of heterologous nucleic acid (for a review, see T. C. Becker et al., Meth. Cell Biol. 43:161-89, 1994; and J. T. Douglas and D. T. Curiel, Science & Medicine 4:44-53, 1997). The adenovirus system offers several advantages: adenovirus can (i) accommodate relatively large DNA inserts; (ii) be grown to high-titer; (iii) infect a broad range of mammalian cell types; and (iv) be used with a large number of available vectors containing different promoters. Also, because adenoviruses are stable in the bloodstream, they can be administered by intravenous injection.

By deleting portions of the adenovirus genome, larger inserts (up to 7 kb) of heterologous DNA can be accommodated. These inserts can be incorporated into the viral DNA by direct ligation or by homologous recombination with a co-transfected plasmid. In an exemplary system, the essential E1 gene has been deleted from the viral vector, and the virus will not replicate unless the E1 gene is provided by the host cell (the human 293 cell line is exemplary). When intravenously administered to intact animals, adenovirus primarily targets the liver. If the adenoviral delivery system has an E1 gene deletion, the virus cannot replicate in the host cells. However, the host's tissue (e.g., liver) will express and process (and, if a secretory signal sequence is present, secrete) the heterologous protein. Secreted proteins will enter the circulation in the highly vascularized liver, and effects on the infected animal can be determined.

Moreover, adenoviral vectors containing various deletions of viral genes can be used in an attempt to reduce or eliminate immune responses to the vector. Such adenoviruses are E1 deleted, and in addition contain deletions of E2A or E4 (Lusky, M. et al., J. Virol. 72:2022-2032, 1998; Raper, S. E. et al., Human Gene Therapy 9:671-679, 1998). In addition, deletion of E2b is reported to reduce immune responses (Amalfitano, A. et al., J. Virol. 72:926-933, 1998). Moreover, by deleting the entire adenovirus genome, very large inserts of heterologous DNA can be accommodated. Generation of so called “gutless” adenoviruses where all viral genes are deleted are particularly advantageous for insertion of large inserts of heterologous DNA. For review, see Yeh, P. and Perricaudet, M., FASEB J. 11:615-623, 1997.

The adenovirus system can also be used for protein production in vitro. By culturing adenovirus-infected non-293 cells under conditions where the cells are not rapidly dividing, the cells can produce proteins for extended periods of time. For instance, BHK cells are grown to confluence in cell factories, then exposed to the adenoviral vector encoding the secreted protein of interest. The cells are then grown under serum-free conditions, which allows infected cells to survive for several weeks without significant cell division. Alternatively, adenovirus vector infected 293S cells can be grown in suspension culture at relatively high cell density to produce significant amounts of protein (see Garnier et al., Cytotechnol. 15:145-55, 1994). With either protocol, an expressed, secreted heterologous protein can be repeatedly isolated from the cell culture supernatant. Within the infected 293S cell production protocol, non-secreted proteins may also be effectively obtained.

As a soluble or cell-surface protein, the activity of zdint5 polypeptide or a peptide to which zdint5 binds, can be measured by a silicon-based biosensor microphysiometer which measures the extracellular acidification rate or proton excretion associated with cell-surface protein interactions and subsequent physiologic cellular responses. An exemplary device is the Cytosensorm™ Microphysiometer manufactured by Molecular Devices, Sunnyvale, Calif. A variety of cellular responses, such as cell proliferation, ion transport, energy production, inflammatory response., regulatory and receptor activation, and the like, can be measured by this method. See, for example, McConnell, H. M. et al., Science 257:1906-1912, 1992; Pitchford, S. et al., Meth. Enzymol. 228:84-108, 1997; Arimilli, S. et al., J. Immunol. Meth. 212:49-59, 1998; Van Liefde, I. et al., Eur. J. Pharmacol. 346:87-95, 1998. The microphysiometer can be used for assaying adherent or non-adherent eukaryotic or prokaryotic cells. By measuring extracellular acidification changes in cell media over time, the microphysiometer directly measures cellular responses to various stimuli, including zdint5 proteins, their agonists, and antagonists. Preferably, the microphysiometer is used to measure responses of a zdint5-responsive eukaryotic cell, compared to a control eukaryotic cell that does not respond to zdint5 polypeptide. zdint5-responsive eukaryotic cells comprise cells into which a polynucleotide for a binding partner for zdint5 has been transfected creating a cell that is responsive to zdint5; or cells naturally responsive to zdint5. Differences, measured by a change in the response of cells exposed to zdint5 polypeptide, relative to a control not exposed to zdint5, are a direct measurement of zdint5-modulated cellular responses. Moreover, such zdint5-modulated responses can be assayed under a variety of stimuli. The present invention provides a method of identifying agonists and antagonists of zdint5 protein, comprising providing cells responsive to a zdint5 polypeptide, culturing a first portion of the cells in the absence of a test compound, culturing a second portion of the cells in the presence of a test compound, and detecting a measurable change in extracellular acidification rate of the second portion of the cells as compared to the first portion of the cells. Moreover, culturing a third portion of the cells in the presence of zdint5 polypeptide and the absence of a test compound provides a positive control for the zdint5-responsive cells, and a control to compare the agonist activity of a test compound with that of the zdint5 polypeptide. Antagonists of zdint5 can be identified by exposing the cells to zdint5 protein in the presence and absence of the test compound, whereby a reduction in zdint5-modulated activity is indicative of agonist activity in the test compound.

Moreover, zdint5 can be used to identify cells, tissues, or cell lines which respond to a zdint5-modulated pathway. The microphysiometer, described above, can be used to rapidly identify cells expressing a zdint5 binding partner, such as cells responsive to zdint5 of the present invention. Cells can be cultured in the presence or absence of zdint5 polypeptide. Those cells which elicit a measurable change in extracellular acidification in the presence of zdint5 are responsive to zdint5. Such cell lines, can be used to identify, antagonists and agonists of zdint5 polypeptide as described above. Using similar methods, cells expressing zdint5 can be used to identify cells which stimulate a zdint5-signalling pathway.

In view of the tissue distribution (colon, small intestine, fetal lung, testis, and B-cells) observed for zdint5 expression, agonists (including the native protease and TSP1-like domains) and antagonists have enormous potential in both in vitro and in vivo applications. Compounds identified as zdint5 agonists and antagonists are useful for studying protease activity, angiogenesis inhibition, extracellular matrix proteins, repair and remodeling of ischemia reperfusion and inflammation in vitro and in vivo. For example, zdint5 and agonist compounds are useful as components of defined cell culture media, and may be used alone or in combination with other cytokines and hormones to replace serum that is commonly used in cell culture. Agonists are thus useful in specifically promoting the growth and/or development of cells of the myeloid and lymphoid lineages in culture. Additionally, zdint5 polypeptides and zdint5 agonists, including small molecules are useful as a research reagent, such as for the expansion, differentiation, and/or protease activity, or angiogenesis inhibition of colon, small intestine, fetal lung, testis, and B-cells. Zdint5 polypeptides are added to tissue culture media for these cell types.

Antagonists are also useful as research reagents for characterizing sites of interactions between members of complement/anti-complement pairs as well as sites of protease activity, or angiogenesis inhibition. Inhibitors of zdint5 activity (zdint5 antagonists) include anti-zdint5 antibodies and soluble zdint5 polypeptides (such as in SEQ ID NOs:2, 5, 8 and 11), as well as other peptidic and non-peptidic agents (including ribozymes).

Zdint5 can also be used to identify inhibitors (antagonists) of its activity. Test compounds are added to the assays disclosed herein to identify compounds that inhibit the activity of zdint5. In addition to those assays disclosed herein, samples can be tested for inhibition of zdint5 activity within a variety of assays designed to measure TSP1-like domain/extracellular matrix binding or the stimulation/inhibition of zdint5-dependent cellular responses. For example, zdint5-modulated cell lines can be transfected with a reporter gene construct that is responsive to a zdint5-stimulated cellular pathway. Reporter gene constructs of this type are known in the art, and will generally comprise a DNA response element operably linked to a gene encoding an assayable protein, such as luciferase, or a metabolite, such as cyclic AMP. DNA response elements can include, but are not limited to, cyclic AMP response elements (CRE), hormone response elements (HRE), insulin response element (IRE) (Nasrin et al., Proc. Natl. Acad. Sci. USA 87:5273-7, 1990) and serum response elements (SRE) (Shaw et al. Cell 56: 563-72, 1989). Cyclic AMP response elements are reviewed in Roestler et al., J. Biol. Chem. 263 (19):9063-6; 1988 and Habener, Molec. Endocrinol. 4 (8):1087-94; 1990. Hormone response elements are reviewed in Beato, Cell 56:335-44; 1989. Candidate compounds, solutions, mixtures or extracts are tested for the ability to inhibit the activity of zdint5 on the target cells, as evidenced by a decrease in zdint5 stimulation of reporter gene expression. Assays of this type will detect compounds that directly block zdint5 binding to a cell-surface protein, i.e., extracellular matrix, or the anti-complementary member of a complementary/anti-complementary pair, as well as compounds that block processes in the cellular pathway subsequent to complement/anti-complement binding. In the alternative, compounds or other samples can be tested for direct blocking of zdint5 binding using zdint5 tagged with a detectable label (e.g., ¹²⁵I, biotin, horseradish peroxidase, FITC, and the like). Within assays of this type, the ability of a test sample to inhibit the binding of labeled zdint5 is indicative of inhibitory activity, which can be confirmed through secondary assays.

Also, zdint5 polypeptides, agonists or antagonists thereof may be therapeutically useful for promoting wound healing, for example, in colon, small intestine, fetal lung, testis, and B-cells tissues. To verify the presence of this capability in zdint5 polypeptides, agonists or antagonists of the present invention, such zdint5 polypeptides, agonists or antagonists are evaluated with respect to their ability to facilitate wound healing according to procedures known in the art. If desired, zdint5 polypeptide performance in this regard can be compared to growth factors, such as EGF, NGF, TGF-α, TGF-β, insulin, IGF-I, IGF-II, fibroblast growth factor (FGF) and the like. In addition, zdint5 polypeptides or agonists or antagonists thereof may be evaluated in combination with one or more growth factors to identify synergistic effects.

A zdint5 polypeptide can also be used for purification of its binding partner(s). The polypeptide is immobilized on a solid support, such as beads of agarose, cross-linked agarose, glass, cellulosic resins, silica-based resins, polystyrene, cross-linked polyacrylamide, or like materials that are stable under the conditions of use. Methods for linking polypeptides to solid supports are known in the art, and include amine chemistry, cyanogen bromide activation, N-hydroxysuccinimide activation, epoxide activation, sulfhydryl activation, and hydrazide activation. The resulting medium will generally be configured in the form of a column, and fluids containing the binding partners are passed through the column one or more times to allow binding partners to bind to the zdint5 polypeptide. The binding partner is then eluted using changes in salt concentration, chaotropic agents (guanidine HCl), or pH to disrupt zdint5/binding partner binding.

An assay system that uses a ligand-binding receptor (or an antibody, one member of a complementary/anti-complementary pair or other cell-surface binding protein) or a binding fragment thereof, and a commercially available biosensor instrument (BIAcore, Pharmacia Biosensor, Piscataway, N.J.) may be advantageously employed. Such receptor, antibody, member of a complement/anti-complement pair or fragment is immobilized onto the surface of a receptor chip. Use of this instrument is disclosed by Karlsson, J. Immunol. Methods 145:229-40, 1991 and Cunningham and Wells, J. Mol. Biol. 234:554-63, 1993. A receptor, antibody, member, disintegrin or fragment is covalently attached, using amine or sulfhydryl chemistry, to dextran fibers that are attached to gold film within the flow cell. A test sample is passed through the cell. If an integrin, epitope, or opposite member of the complementary/anti-complementary pair is present in the sample, it will bind to the immobilized disintegrin, antibody or member, respectively, causing a change in the refractive index of the medium, which is detected as a change in surface plasmon resonance of the gold film. This system allows the determination of on- and off-rates, from which binding affinity can be calculated, and assessment of.

Protease substrate polypeptides and TSP1-like binding polypeptides which bind proteases or TSP1-like polypeptides can also be used within other assay systems known in the art. Similarly, extracellular matrix polypeptides which bind to the TSP-like polypeptides of zdint5 can also be used with other assay systems. Such systems include Scatchard analysis for determination of binding affinity (see Scatchard, Ann. NY Acad. Sci. 51: 660-72, 1949) and calorimetric assays (Cunningham et al., Science 253:545-48, 1991; Cunningham et al., Science 245:821-25, 1991).

A “soluble protein” is a protein that is not bound to a cell membrane. Soluble proteins are most commonly ligand-binding receptor polypeptides that lack transmembrane and cytoplasmic domains. Soluble proteins can comprise additional amino acid residues, such as affinity tags that provide for purification of the polypeptide or provide sites for attachment of the polypeptide to a substrate, or immunoglobulin constant region sequences. Many cell-surface proteins have naturally occurring, soluble counterparts that are produced by proteolysis or translated from alternatively spliced mRNAs. Proteins are said to be substantially free of transmembrane and intracellular polypeptide segments when they lack sufficient portions of these segments to provide membrane anchoring or signal transduction, respectively.

Soluble forms of zdint5 polypeptides, such as the polypeptide of SEQ ID NOs:2, 5, 8 and 11, may act as antagonsits to or agonists of zdint5 polypeptides, and would be useful to modulate the effects of zdint5 in colon, small intestine, fetal lung, testis, and B-cells.

Molecules of the present invention can be used to identify and isolate extracellular matrix proteins, or members of complement/anti-complement pairs involved in protease activity, or angiogenesis inhibition. For example, proteins and peptides of the present invention can be immobilized on a column and membrane preparations run over the column (Immobilized Affinity Ligand Techniques, Hermanson et al., eds., Academic Press, San Diego, Calif., 1992, pp.195-202). Proteins and peptides can also be radiolabeled (Methods in Enzymol., vol. 182, “Guide to Protein Purification”, M. Deutscher, ed., Acad. Press, San Diego, 1990, 721-37) or photoaffinity labeled (Brunner et al., Ann. Rev. Biochem. 62:483-514, 1993 and Fedan et al., Biochem. Pharmacol. 33:1167-80, 1984) and specific cell-surface proteins can be identified.

The polypeptides, nucleic acid and/or antibodies of the present invention can be used in treatment of disorders associated with recovery after gastrointestinal irradiation, chemotherapy, or antibody use. Additionally, molecules of the present invention may be useful as anti-infectives, and/or extracellular matrix repair and remodeling. The molecules of the present invention can be used to modulate proteolysis, apoptosis, angiogenesis, infection, cell adhesion, cell fusion, and signaling or to treat or prevent development of pathological conditions in such diverse tissue as colon, small intestine, fetal lung, testis, and B-cells. In particular, certain diseases may be amenable to such diagnosis, treatment or prevention. The molecules of the present invention can be used to modulate inhibition and proliferation of endothelium in colon, small intestine, fetal lung, testis, and B-cells. Disorders which may be amenable to diagnosis, treatment or prevention with zdint5 polypeptides include, for example, tumor formation, Crohn's Disease, Inflammatory Bowel Disease, food poisoning, melanoma, and degenerative diseases.

Polynucleotides encoding zdint5 polypeptides are useful within gene therapy applications where it is desired to increase or inhibit zdint5 activity. If a mammal has a mutated or absent zdint5 gene, the zdint5 gene can be introduced into the cells of the mammal. In one embodiment, a gene encoding a zdint5 polypeptide is introduced in vivo in a viral vector. Such vectors include an attenuated or defective DNA virus, such as, but not limited to, herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes simplex virus 1 (HSV1) vector (Kaplitt et al., Molec. Cell. Neurosci. 2:320-30, 1991); an attenuated adenovirus vector, such as the vector described by Stratford-Perricaudet et al., J. Clin. Invest. 90:626-30, 1992; and a defective adeno-associated virus vector (Samulski et al., J. Virol. 61:3096-101, 1987; Samulski et al., J. Virol. 63:3822-8, 1989).

In another embodiment, a zdint5 gene can be introduced in a retroviral vector, e.g., as described in Anderson et al., U.S. Pat. No. 5,399,346; Mann et al. Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; International Patent Publication No. WO 95/07358, published Mar. 16, 1995 by Dougherty et al.; and Kuo et al., Blood 82:845, 1993. Alternatively, the vector can be introduced by lipofection in vivo using liposomes. Synthetic cationic lipids can be used to prepare liposomes for in vivo transfection of a gene encoding a marker (Felgner et al., Proc. Natl. Acad. Sci. USA 84:7413-7, 1987; Mackey et al., Proc. Natl. Acad. Sci. USA 85:8027-31, 1988). The use of lipofection to introduce exogenous genes into specific organs in vivo has certain practical advantages. Molecular targeting of liposomes to specific cells represents one area of benefit. More particularly, directing transfection to particular cells represents one area of benefit. For instance, directing transfection to particular cell types would be particularly advantageous in a tissue with cellular heterogeneity, such as the pancreas, liver, kidney, and brain. Lipids may be chemically coupled to other molecules for the purpose of targeting. Targeted peptides (e.g., hormones or neurotransmitters), proteins such as antibodies, or non-peptide molecules can be coupled to liposomes chemically.

Similarly, the zdint5 polynucleotides (SEQ ID NOs:1, 3, 4, 6, 7, 9, 10, or 12) can be used to target specific tissues such as colon, small intestine, fetal lung, testis, and B-cells. It is possible to remove the target cells from the body; to introduce the vector as a naked DNA plasmid; and then to re-implant the transformed cells into the body. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter. See, e.g., Wu et al., J. Biol. Chem. 267:963-7, 1992; Wu et al., J. Biol. Chem. 263:14621-4, 1988.

Various techniques, including antisense and ribozyme methodologies, can be used to inhibit zdint5 gene transcription and translation, such as to inhibit cell proliferation in vivo. Polynucleotides that are complementary to a segment of a zdint5-encoding polynucleotide (e.g., a polynucleotide as set forth in SEQ ID NOs: 1, 3, 4, 6, 7, 9, 10 or 12) are designed to bind to zdint5-encoding mRNA and to inhibit translation of such mRNA. Such antisense polynucleotides are used to inhibit expression of zdint5 polypeptide-encoding genes in cell culture or in a subject.

Mice engineered to express the zdint5 gene, referred to as “transgenic mice,” and mice that exhibit a complete absence of zdint5 gene function, referred to as “knockout mice,” may also be generated (Snouwaert et al., Science 257:1083, 1992; Lowell et al., Nature 366:740-42, 1993; Capecchi, M. R., Science 244: 1288-1292, 1989; Palmiter, R. D. et al. Annu Rev Genet. 20: 465-499, 1986). For example, transgenic mice that over-express zdint5, either ubiquitously or under a tissue-specific or tissue-restricted promoter can be used to ask whether over-expression causes a phenotype. For example, over-expression of a wild-type zdint5 polypeptide, polypeptide fragment or a mutant thereof may alter normal cellular processes, resulting in a phenotype that identifies a tissue in which zdint5 expression is functionally relevant and may indicate a therapeutic target for the zdint5, its agonists or antagonists. For example, a transgenic mouse to engineer is one that over-expresses the soluble zdint5 polypeptide (approximately amino acids 104 to 306 of SEQ ID NO:11). Moreover, such over-expression may result in a phenotype that shows similarity with human diseases. Similarly, knockout zdint5 mice can be used to determine where zdint5 is absolutely required in vivo. The phenotype of knockout mice is predictive of the in vivo effects of that a zdint5 antagonist, such as those described herein, may have. The human zdint5 cDNA can be used to isolate murine zdint5 mRNA, cDNA and genomic DNA, which are subsequently used to generate knockout mice. These mice may be employed to study the zdint5 gene and the protein encoded thereby in an in vivo system, and can be used as in vivo models for corresponding human diseases. Moreover, transgenic mice expression of zdint5 antisense polynucleotides or ribozymes directed against zdint5, described herein, can be used analogously to transgenic mice described above.

Zdint5 polypeptides, variants, and fragments thereof, may be useful as replacement therapy for disorders associated with protease activity, or angiogenesis inhibition, including disorders related to, for example, immuntiy, inflammation, fertility, gamete maturation, immunology, trauma, and epithelial disorders, in general.

A less widely appreciated determinant of tissue morphogenesis is the process of cell rearrangement: Both cell motility and cell-cell adhesion are likely to play central roles in morphogenetic cell rearrangements. Cells need to be able to rapidly break and probably simultaneously remake contacts with neighboring cells. See Gumbiner, B. M., Cell 69:385-387, 1992. As a secreted protein in colon, small intestine, fetal lung, testis, and B-cells, zdint5 can play a role in intercellular rearrangement in these and other tissues.

The zdint5 polypeptide is expressed in tissues of the colon, small intestine, testis, lung and B cells. Thus, the polypeptides of the present invention are useful in studying cell adhesion and the role thereof in metastasis and may be useful in preventing metastasis, in particular metastasis in tumors of the colon, small intestine, testis, lung and B cells. Similarly, polynucleotides and polypeptides of zdint5 may be used to replace their defective counterparts in tumor or diseased tissues. Thus, zdint5 polypeptide pharmaceutical compositions of the present invention may be useful in prevention or treatment of disorders associated with pathological regulation or the expansion of these tissues. The polynucleotides of the present invention may also be used in conjunction with a regulatable promoter, thus allowing the dosage of delivered protein to be regulated.

Moreover, the activity and effect of zdint5 on tumor progression and metastasis can be measured in vivo. Several syngeneic mouse models have been developed to study the influence of polypeptides, compounds or other treatments on tumor progression. In these models, tumor cells passaged in culture are implanted into mice of the same strain as the tumor donor. The cells will develop into tumors having similar characteristics in the recipient mice, and metastasis will also occur in some of the models. Tumor models include the Lewis lung carcinoma (ATCC No. CRL-1642) and B16 melanoma (ATCC No. CRL-6323), amongst others. These are both commonly used tumor lines, syngeneic to the C57BL6 mouse, that are readily cultured and manipulated in vitro. Tumors resulting from implantation of either of these cell lines are capable of metastasis to the lung in C57BL6 mice. The Lewis lung carcinoma model has recently been used in mice to identify an inhibitor of angiogenesis (O'Reilly M S, et al. Cell 79: 315-328,1994). C57BL6/J mice are treated with an experimental agent either through daily injection of recombinant protein, agonist or antagonist or a one time injection of recombinant adenovirus. Three days following this treatment, 10⁵ to 10⁶ cells are implanted under the dorsal skin. Alternatively, the cells themselves may be infected with recombinant adenovirus, such as one expressing zdint5, before implantation so that the protein is synthesized at the tumor site or intracellularly, rather than systemically. The mice normally develop visible tumors within 5 days. The tumors are allowed to grow for a period of up to 3 weeks, during which time they may reach a size of 1500-1800 mm in the control treated group. Tumor size and body weight are carefully monitored throughout the experiment. At the time of sacrifice, the tumor is removed and weighed along with the lungs and the liver. The lung weight has been shown to correlate well with metastatic tumor burden. As an additional measure, lung surface metastases are counted. The resected tumor, lungs and liver are prepared for histopathological examination, immunohistochemistry, and in situ hybridization, using methods known in the art and described herein. The influence of the expressed polypeptide in question, e.g., zdint5, on the ability of the tumor to recruit vasculature and undergo metastasis can thus be assessed. In addition, aside from using adenovirus, the implanted cells can be transiently transfected with zdint5. Moreover, purified zdint5 or zdint5-conditioned media can be directly injected in to this mouse model, and hence be used in this system. Use of stable zdint5 transfectants as well as use of induceable promoters to activate zdint5 expression in vivo are known in the art and can be used in this system to assess zdint5 induction of metastasis. For general reference see, O'Reilly MS, et al. Cell 79:315-328, 1994; and Rusciano D, et al. Murine Models of Liver Metastasis. Invasion Metastasis 14:349-361, 1995.

Zdint5 gene may be useful to as a probe to identify humans who have a defective zdint5 gene. The strong expression of zdint5 in colon, small intestine, fetal lung, testis, and B-cells suggests that zdint5 polynucleotides or polypeptides can be used as measured as an indication of aberrant growth in these tissues. Thus, polynucleotides and polypeptides of zdint5, and mutations to them, can be used a diagnostic indicators of cancer in these tissues.

The polypeptides of the present invention are useful in studying cell adhesion and the role thereof in metastasis and may be useful in preventing metastasis, in particular metastasis in tumors of the colon, small intestine, fetal lung, testis, and B-cells. Similarly, polynucleotides and polypeptides of zdint5 may be used to replace their defective counterparts in tumor or malignant tissues.

The zdint5 polypeptide is expressed in the colon, small intestine, fetal lung, testis, and B-cells. Thus, zdint5 polypeptide pharmaceutical compositions of the present invention may be useful in prevention or treatment of disorders associated with pathological regulation or the expansion of colon, small intestine, fetal lung, testis, and B-cells.

The zdint5 polynucleotides of SEQ ID NOs:2, 5, 8, and 11 have been mapped to chromosome 9q34. Thus, the present invention also provides reagents which will find use in diagnostic applications. For example, the zdint5 gene, a probe comprising zdint5 DNA or RNA or a subsequence thereof can be used to determine if the zdint5 gene is present on chromosome 9q34 or if a mutation has occurred. Detectable chromosomal aberrations at the zdint5 gene locus include, but are not limited to, aneuploidy, gene copy number changes, insertions, deletions, restriction site changes and rearrangements. Such aberrations can be detected using polynucleotides of the present invention by employing molecular genetic techniques, such as restriction fragment length polymorphism (RFLP) analysis, short tandem repeat (STR) analysis employing PCR techniques, and other genetic linkage analysis techniques known in the art (Sambrook et al., ibid.; Ausubel et. al., ibid.; Marian, Chest 108:255-65, 1995).

For pharmaceutical use, the proteins of the present invention can be administered orally, rectally, parenterally (particularly intravenous or subcutaneous), intracistemally, intravaginally, intraperitoneally, topically (as powders, ointments, drops or transdermal patch) bucally, in utero or as a pulmonary or nasal inhalant. Intravenous administration will be by bolus injection or infusion over a typical period of one to several hours. In general, pharmaceutical formulations will include a zdint5 protein, alone, or in conjunction with a dimeric partner, in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19th ed., 1995. Therapeutic doses will generally be in the range of 0.1 to 100 μg/kg of patient weight per day, preferably 0.5-20 mg/kg per day, with the exact dose determined by the clinician according to accepted standards, taking into account the nature and severity of the condition to be treated, patient traits, etc. Determination of dose is within the level of ordinary skill in the art. The proteins may be administered for acute treatment, over one week or less, often over a period of one to three days or may be used in chronic treatment, over several months or years. In general, a therapeutically effective amount of zdint5 is an amount sufficient to produce a clinically significant change in extracellular matrix remodeling, scar tissue formation, tumor suppression, platelet aggregation, apoptosis, myogenesis, colon, small intestine, fetal lung, testis, and B-cells tissues. Similarly, a therapeutically effective amount of zdint5 is an amount sufficient to produce a clinically significant change in disorders associated with colon, small intestine, lung, testis, and B-cells.

What is claimed is:

1. An isolated polypeptide comprising the amino acid sequence as shown in SEQ ID NO:2.

2. The isolated polypeptide according to claim 1, further comprising the amino acid sequence as shown in SEQ ID NO:11.

3. The isolated polypeptide according to claim 1 wherein the polypeptide is operably linked via a peptide bond or polypeptide linker to a second polypeptide selected from the group consisting of maltose binding protein, an immunoglobulin constant region, and a polyhistidine tag.

4. An isolated polynucleotide encoding a fusion protein comprising a first polypeptide segment and a second polypeptide segment, wherein the first polypeptide segment comprises the polypeptide according to claim 1, and the second polynucleotide segment encodes a second polypeptide that encodes one or more TSP1-like domain, and wherein the first polynucleotide segment is positioned amino-terminally to the second polynucleotide segment.

5. An isolated polypeptide comprising the amino acid sequence selected fro the group consisting of
a) a polypeptide comprising the amino acid sequence as shown in SEQ ID NO:5; and
b) a polypeptide comprising the amino acid sequence as shown in SEQ ID NO8.

6. The isolated polypeptide according to claim 5 wherein the polypeptide is operably linked via a peptide bond or polypeptide linker to a second polypeptide selected from the group consisting of maltose binding protein, an immunoglobulin constant region, and a polyhistidine tag.

7. An isolated polynucleotide encoding a fusion protein comprising a first polypeptide segment and a second polypeptide segment, wherein the first polypeptide segment comprises a protease domain and the second polypeptide segment comprises one or more polypeptides selected from the group consisting of:
(a) a polypeptide comprising residues 1 to 48 of SEQ ID NO:5; and
(b) a polypeptide comprising residues 1 to 59 of SEQ ID NO:8;
wherein the first polypeptide segment is positioned amino-terminally to the second polypeptide segment.

8. An expression vector comprising the following operably linked elements:
a) a transcription promoter;
b) a DNA segment encoding a polypeptide, wherein the amino acid sequence of the polypeptide comprises the amino acid sequence selected from the group consisting of:
i) the amino acid sequence as shown in SEQ ID NO:2;
ii) the amino acid sequence as shown in SEQ ID NO:5;
iii) the amino acid sequence as shown in SEQ ID NO:8; and
iv) the amino acid sequence as shown in SEQ ID NO:11; and
c) a transcription terminator.

9. The expression vector according to claim 8, further linked to an affinity tag.

10. A cultured cell into which has been introduced an expression vector according to claim 8, wherein said cell expresses the polypeptide encoded by the DNA segment.

11. A method of producing a polypeptide comprising culturing a cell according to claim 10, whereby said cell expresses the polypeptide encoded by the DNA segment, and recovering the polypeptide.

12. The polypeptide made by the method of claim 11.

13. A method for modulating extracellular matrix interactions by combining the polypeptide according to claim 12 with cells.

14. A method for modulating extracellular matrix interactions according to claim 13, whereby the cells are derived from tissues selected from the group consisting of:
a) tissues from colon;
b) tissues from small intestine;
c) tissues from testes; and
d) tissues from lung.

15. An antibody the specifically binds to a polypeptide consisting of the amino acid sequence as shown in SEQ ID NO:11.

16. The antibody according to claim 15, wherein the antibody specifically binds to a polypeptide consisting of the amino acid sequence as shown in SEQ ID NO:2.

17. The antibody according to claim 15, wherin the antibody specifically binds to a polypeptide selected from the group consisting of:
a) a polypeptide consisting of the amino acid sequence as shown in SEQ ID NO:5; and
b) a polypeptide consisting of the amino acid sequence as shown in SEQ ID NO:8.

18. A method of producing an antibody to the polypeptide according to claim 12 comprising the following steps:
inoculating an animal with the polypeptide such that the polypeptide elicits an immune response in the animal to produce the antibody; and
isolating the antibody from the animal.

19. An antibody produced by the method of claim 18, wherein the antibody specifically binds to a polypeptide selected from the group consisting of:
a) a polypeptide comprising the amino acid sequence as shown in SEQ ID NO:2;
b) a polypeptide comprising the amino acid sequence as shown in SEQ ID NO:5;
c) a polypeptide comprising the amino acid sequence as shown in SEQ ID NO8; and
d) a polypeptide comprising the amino acid sequence as shown in SEQ ID NO11.

20. An isolated immunogenic polypeptide, the amino acid sequenc of which comprises at least 30 contiguous amino acids of SEQ ID NO:11.
